(19) United States
(12) Patent Application Publication
(10) Pub. No.: US 2026/0363876 A1
(43) Pub. Date: Nov. 19, 2026

(54) IMPLANT AGNOSTIC COMPUTER-ASSISTED SURGICAL SYSTEM, APPARATUS, AND METHOD

(71) Applicant: Think Surgical, Inc., Fremont, CA (US)

(72) Inventors: Jonathan Gibson, Fremont, CA (US); Eytan M. Debbi, Bronx, NY (US); Joel F. Zuhars, Dublin, CA (US)

(21) Appl. No.: 19/573,717

(22) Filed: Mar. 20, 2026

Related U.S. Application Data
(63) Continuation of application No. PCT/US24/49974, filed on Oct. 4, 2024.
(60) Provisional application No. 63/542,624, filed on Oct. 5, 2023.
(60) Provisional application No. 63/657,517, filed on Jun. 7, 2024.

(51) Int. Cl. A61F 2/46; A61B 34/00; A61B 34/10

(52) U.S. Cl. CPC A61F 2/461; A61B 34/10; A61B 34/25; A61B 2034/102; A61B 2034/107; A61B 2034/108; A61B 2034/254

(57) ABSTRACT

In TKA, after the posterior cut surface is formed, a spacer is placed on the posterior cut surface (e.g., directly thereon, or sandwiched between the posterior cut surface and the tibia plateau or tibia cut surface) to replace the removed posterior condyles in order to use the manufacturer's sizing guide to: (i) determine an appropriate implant size; and/or (ii) determine the location for mounting the 4-in-1 block on the distal cut surface to align the 4-in-1 cut block in the correct POSE to guide the formation of the remaining cut surfaces to accommodate the determined implant size. The thickness of the spacer may replicate (or matches) the thickness of the femoral implant posterior thickness or the thickness of the removed posterior condyles. The spacer may have an adjustment mechanism to account for the posterior thickness of different implant lines or the thickness of the removed posterior condyles.

### CROSS-REFERENCE TO RELATED APPLICATION(S)

This patent application is a continuation of International Patent Application No. PCT/US2024/049974 entitled IMPLANT AGNOSTIC COMPUTER-ASSISTED SURGICAL SYSTEM, APPARATUS, AND METHOD filed Oct. 4, 2024, which claims priority from U.S. Provisional Patent Application No. 63/657,517 entitled IMPLANT AGNOSTIC COMPUTER-ASSISTED SURGICAL SYSTEM, APPARATUS, AND METHOD filed Jun. 7, 2024 and from U.S. Provisional Patent Application No. 63/542,624 entitled IMPLANT AGNOSTIC COMPUTER-ASSISTED SURGICAL SYSTEM, APPARATUS, AND METHOD filed Oct. 5, 2023, each of which is hereby incorporated herein by reference in its entirety. The subject matter of this patent application also may be related to the subject matter of commonly-owned PCT Application No. PCT/US2022/046146 filed Oct. 10, 2022 and published as PCT Publication No. WO/2023/059931 on Apr. 13, 2023, which claims priority from U.S. Provisional Patent Application No. 63/253,923 filed Oct. 8, 2021, each of which is hereby incorporated herein by reference in its entirety.

### FIELD OF THE INVENTION

The invention generally relates to computer-assisted surgical devices.

### BACKGROUND OF THE INVENTION

In a conventional Total Knee Arthroplasty (TKA), commonly referred to as a total knee replacement, a distal femoral cut is made to form a distal cut surface on the femur. A sizing guide is then mounted to the distal cut surface to assist the surgeon in determining an appropriate implant size for the implant and to determine the desired internal-external (I-E) rotation for mounting the implant on the femur. The sizing guide, once properly positioned according to the determined implant size and I-E rotation, includes holes for guiding a drill bit to form holes in the bone, where the drilled holes receive pegs of a 4-in-1 cut block to mount a 4-in-1 cut block on the distal cut surface in the proper alignment to guide the formation of the remaining cut surfaces on the distal femur.

More specifically, throughout a lifetime, bones and joints become damaged and worn through normal use, disease, and traumatic events. Arthritis is a leading cause of joint damage that over time leads to cartilage degradation, pain, stiffness, and bone loss. Arthritis can also cause the muscles articulating the joints to lose strength and become painful.

If the pain associated with the dysfunctional joint is not alleviated by less-invasive therapies, a joint arthroplasty procedure is considered as a treatment. Joint arthroplasty is an orthopedic procedure in which an arthritic or dysfunctional joint surface is replaced with an orthopedic implant.

The accurate placement and alignment of an implant is a large factor in determining the success of joint arthroplasty. In total knee arthroplasty (TKA), the articulating surfaces of the knee joint are replaced with prosthetic components, or implants, typically formed of metal or plastic, to create new articulating joint surfaces. The implants include surfaces intended to contact bone. TKA requires the removal of worn or damaged articular cartilage and bone on the distal femur and proximal tibia, where the removal cuts the bone to provide surfaces (“cut surfaces”) on the remaining bone to contact the contact surfaces of the implant. The position and orientation (POSE) of the cut surfaces determine the final placement and POSE of the implants within the joint. Generally, surgeons plan and create the cut surfaces so the final placement of the implants restores the mechanical axis or kinematics of the patient's leg while preserving the balance of the surrounding knee ligaments. Even small implant alignment errors outside of clinically acceptable ranges correlate with worse outcomes and increased rates of revision surgery.

Current TKA implants are designed to be installed using specific manual instrumentation (e.g., cutting jigs, cutting blocks, alignment fixtures) to form the cut surfaces. Femoral implants typically have five femoral contact surfaces and one or more stabilizing features (e.g., pegs, boxes). The five femoral contact surfaces are intended to contact five cut surfaces on the remaining femur. The stabilizing features of a femoral implant may include pegs or a box to stabilize the femoral implant on the femur. The pegs or box are intended to be inserted into stabilizing cut features (e.g., holes) cut into the bone, typically through a cut surface of the femur and are typically formed perpendicular to a cut surface. Tibial implants typically have one tibial contact surface and a stabilizing feature (e.g., a keel). The one tibial contact surface is intended to contact one cut surface on the remaining tibia. The stabilizing features of a tibial implant may include a keel to stabilize the tibial implant on the tibia. The keel is intended to be inserted into a stabilizing cut feature (e.g., a hole) that is cut into the bone, typically through the cut surface of the tibia, and is typically formed perpendicular to the cut surface. FIGS. 1A-1C illustrate a patient's distal femur 10 and a contour matching femoral implant 12 for a TKA procedure, where five contact surfaces on the implant are intended to contact five cut surfaces on the femur. The anterior cut surface 14 is intended to contact the anterior contact surface 13, the anterior chamfer cut surface 16 is intended to contact the anterior chamfer contact surface 15, the distal cut surface 18 is intended to contact the distal contact surface 17, the posterior chamfer cut surface 20 is intended to contact the posterior chamfer contact surface 19, and the posterior cut surface 22 is intended to contact the posterior contact surface 21. The femoral implant 12 also includes stabilizing features in the form of pegs (23, 24) intended to be inserted into stabilizing cut features (e.g., holes, not shown) formed into the distal cut surface 18 of the femur 10. The manual instrumentation is aligned and affixed to the bone to guide the formation of each cut surface. One of the key struggles associated with manual TKA is the proper alignment of the manual instrumentation on the bone. The user has to reference various anatomical landmarks and adjust the cutting jigs based on measurements to ensure the instruments are properly aligned. Aligning the cutting jigs to assist the user in forming these cut surfaces is particularly difficult.

After the femoral distal cut surface is formed on the bone, a sizing guide 25 is mounted to the distal cut surface 18 as shown in FIG. 2 to assist the surgeon in determining an appropriate implant size for the implant and a desired internal-external rotation for mounting the implant on the bone. The sizing guide 25 includes guide holes 26 that receive and guide a drill bit to form holes in the bone (or for guiding the placement of pins in the bone). The sizing guide 25 may further include paddles 27 that reference (or rest against) the posterior condyles ‘PC’ to assist with the alignment. Once the sizing guide 25 is properly positioned on the bone according to the determined implant size and I-E rotation, holes are drilled into the bone by advancing a drill bit through the guide holes 26. The drilled holes are then used to mount a 4-in-1 cut block 40 (as shown in FIGS. 3A and 3B) on the distal cut surface in the proper alignment to guide the formation of the remaining cut surfaces on the bone. The remaining cut surfaces are easily formed by advancing a surgical saw blade through guide slots of the 4-in-1 block.

FIGS. 3A and 3B depict a conventional 4-in-1 cut block used in TKA procedures, where FIG. 3A is top perspective view thereof, and FIG. 3B is bottom perspective view thereof. The 4-in-1 cut block 40 may include a body 42 having a top surface 44, a bottom surface 45, a pair of pegs (54a, 54b) extending from the bottom surface 45, and a plurality of guide slots. The plurality of guide slots may include an anterior guide slot 52, an anterior chamfer guide slot 50, a posterior chamfer guide slot 48, and a posterior guide slot 46. Each of the guide slots receive a surgical saw blade for guiding the surgical saw blade in forming the corresponding cut surfaces on the bone. The pegs (54a, 54b) are configured to fit in the peg holes formed on the femoral distal cut surface 18. The peg holes may have been formed on the distal cut surface 18 by advancing a drill bit through the guide holes 26 in the manufacturer's sizing guide 25 as previously described.

Computer-assisted surgery (CAS) can overcome many of the obstacles associated with manual instrumentation. Computer-assisted surgery is an expanding field having applications in total joint arthroplasty (TJA), unicondylar knee arthroplasty, bone fracture repair, maxillofacial reconstruction, and spinal reconstruction. CAS systems (e.g., surgical robotic systems) are particularly useful for surgical procedures requiring dexterity, precision, and accuracy, and generally include planning software and a computer-assisted surgical device. Examples of these CAS devices are the RIO® Robotic Arm (Stryker/Mako), the TSolution One® Surgical System (THINK Surgical), the TMINI® Miniature Robotic System (THINK Surgical), and the ROSA® Surgical System (Zimmer). All of these systems generally include planning software for planning the location of an implant with respect to the bone. The planning software may include 3-D models of the patient's bones, 3-D models of various implants, and software tools that allow the user to position the implant models relative to the bone models. The CAS device then executes the plan to assist in forming the cut surfaces on the bones to mount the implants thereon in the planned POSE.

During the planning phase, the planning software is used to build a 3-D bone model of the patient's bone using pre-operative images (e.g., computed tomography (CT) scans, magnetic resonance imaging (MRI)). The planning software includes an implant library having a plurality of implant models that may be positioned relative to the 3-D bone models to designate the best fit, fill, and/or alignment of the implants on the bones. A user may therefore select an implant model of their choice in the implant library and plan the procedure accordingly.

All of the implants available in the implant library are supported by the CAS system. The CAS system uses the known geometry of each supported implant to accurately cut the bone in the proper shape, position, and orientation to form the cut surfaces on the bone corresponding to the implant contact surfaces to allow the implant to be mounted to the bone in the planned position and orientation.

However, surgeons have widely different preferences on the types of implants they routinely use. If the CAS system does not support a particular implant (e.g., the full geometry data of the implant is unavailable), then a surgeon cannot use that implant with the CAS system. Likewise, different implant lines may be more beneficial to one patient than another. A patient cannot benefit from the use of the CAS system if the implant needed for the patient is not supported by the CAS system.

### SUMMARY OF VARIOUS EMBODIMENTS

In accordance with one embodiment of the invention, a method for computer-assisted surgery involves using a computer-assisted surgical (CAS) device to assist in forming a distal cut surface and a posterior cut surface on a femur bone for a total knee arthroplasty (TKA) procedure, wherein forming the posterior cut surface removes the posterior condyles needed for sizing an implant with an implant manufacture's sizing guide; and placing a spacer on the posterior cut surface (e.g., directly thereon, or sandwiched between the posterior cut surface and the tibia plateau or tibia cut surface) to replace the removed posterior condyles in order to use the implant manufacturer's sizing guide.

In various alternative embodiments, the user may be instructed to mount the implant manufacturer's sizing guide on the distal cut surface with the spacer in place, and then, after mounting of the implant manufacturer's sizing guide, the mounted implant manufacturer's sizing guide may be used to determine an implant size from a plurality of manufacturer implant sizes. Then, holes in the mounted implant manufacturer's sizing guide may be used to insert pins or drill holes in the bone. Then, the pins or holes may be used to align a 4-in-1 cut block in a correct POSE to form remaining cut surfaces to accommodate the determined implant size. The spacer may be an adjustable thickness spacer (e.g., adjustable between at least 8 mm and 11 mm such as in 0.5 mm increments) or the spacer may be a fixed thickness spacer. The thickness of the spacer may be equal to or adjustable to a posterior thickness of a femoral implant or to the thickness of the removed posterior condyles. The CAS device may assist in the formation of the distal cut surface and the posterior cut surface without having implant geometry data for the implant. The method also may involve insertion of the pins, wherein the pins may be tiered alignment pins having a bottom portion with a diameter greater than a top portion and/or the pins may be inserted using an alignment pin assembly having a sleeve and a pin.

In accordance with another embodiment, a system for computer-assisted surgery comprises a computer-assisted surgical (CAS) device for assisting in the formation of a distal cut surface and a posterior cut surface on a femur bone during a total knee arthroplasty (TKA) procedure, wherein forming the posterior cut surface removes the posterior condyles needed for sizing an implant with an implant manufacture's sizing guide; and a spacer for placement on the posterior cut surface to replace the removed posterior condyles in order to use the implant manufacturer's sizing guide.

In various alternative embodiments, the system may further include the implant manufacturer's sizing guide for mounting to the distal cut surface with the spacer in place. The implant manufacturer's sizing guide may be used to determine an implant size from a plurality of manufacturer implant sizes. For example, the implant manufacturer's sizing guide may include guide holes for guiding the placement of pins in the bone or for drilling holes in the bone, wherein the pins or holes are used to align a 4-in-1 cut block in a correct POSE to form remaining cut surfaces to accommodate the determined implant size. The spacer may be an adjustable thickness spacer (e.g., adjustable between at least 8 mm and 11 mm such as in 0.5 mm increments) or the spacer may be a fixed thickness spacer. The thickness of the spacer may be equal to or adjustable to a posterior thickness of a femoral implant or to the thickness of the removed posterior condyles. The CAS device may assist in the formation of the distal cut surface and the posterior cut surface without having implant geometry data for the implant. The pins may be inserted, wherein the pins may be tiered alignment pins having a bottom portion with a diameter greater than a top portion and/or the pins may be inserted using an alignment pin assembly having a sleeve and a pin.

In accordance with another embodiment, a spacer is specifically configured for placement on a posterior cut surface to replace removed posterior condyles needed to mount an implant manufacture's sizing guide on a distal cut surface for a total knee arthroplasty (TKA) procedure. The spacer may be an adjustable thickness spacer (e.g., adjustable between at least 8 mm and 11 mm such as in 0.5 mm increments) or the spacer may be a fixed thickness spacer. The thickness of the spacer may be equal to or adjustable to a posterior thickness of a femoral implant or to the thickness of the removed posterior condyles. The spacer may be configured to by referenced by paddles or other posterior condyle referencing feature of the sizing guide.

In accordance with another embodiment, a CAS planning system comprises at least one processor and at least one tangible, non-transitory computer-readable medium having embodied therein computer program instructions, which, when executed by the at least one processor, implement an implant agnostic graphical user interface (GUI) for defining a distal cut surface and a posterior cut surface for a total knee arthroplasty (TKA) procedure without having implant geometry data for a manufacturer's implant.

In various alternative embodiments, the implant agnostic GUI may provide at least one of: an interface displaying a graphic (e.g., line, box, grayed out bone areas) showing the location of a planned bone cut; an interface showing a generic implant model as a visualization tool to assist in planning the location for the implant relative to the bone; an interface displaying alignment information comprising at least one of degrees of varus-valgus of the bone from the mechanical axis as a result of a planned distal cut, the degrees of external-internal rotation of the implant from a condylar axis (e.g., transepicondylar axis (TEA), posterior condylar axis (PCA)) as a result of a planned posterior cut, the degrees of flexion-extension of the implant relative to the bone as a result of a planned distal cut or planned posterior cut, or the posterior slope of the implant relative to bone as a result of the planned tibial cut; or an interface displaying an amount of bone that will be resected for a planned bone cut.

In accordance with another embodiment, a computer program product comprising at least one tangible, non-transitory computer-readable medium having embodied therein computer program instructions which, when executed by at least one processor, implement an implant agnostic graphical user interface (GUI) for defining a distal cut surface and a posterior cut surface for a total knee arthroplasty (TKA) procedure without having implant geometry data for a manufacturer's implant.

In various alternative embodiments, the implant agnostic GUI may provide at least one of: an interface displaying a graphic (e.g., line, box, grayed out bone areas) showing the location of a planned bone cut; an interface showing a generic implant model as a visualization tool to assist in planning the location for the implant relative to the bone; an interface displaying alignment information comprising at least one of degrees of varus-valgus of the bone from the mechanical axis as a result of a planned distal cut, the degrees of external-internal rotation of the implant from a condylar axis (e.g., transepicondylar axis (TEA), posterior condylar axis (PCA)) as a result of a planned posterior cut, the degrees of flexion-extension of the implant relative to the bone as a result of a planned distal cut or planned posterior cut, or the posterior slope of the implant relative to bone as a result of the planned tibial cut; or an interface displaying an amount of bone that will be resected for a planned bone cut.

In accordance with another embodiment, a system for computer-assisted surgery comprises a computer-assisted surgical (CAS) device for assisting in the formation of a distal cut surface and a posterior cut surface on a femur bone during a total knee arthroplasty (TKA) procedure; and an alignment guide configured for placement on the posterior cut surface to align a posterior guide slot of a cut block with the location of the posterior cut surface.

In various alternative embodiments, the alignment guide may include one or more alignment features configured to fit inside the posterior guide slot and/or a face for placement against the posterior cut surface. The alignment guide may be at least one of a surgical saw blade or a retractor. The thickness of the alignment feature may be less than a size of an opening of the posterior guide slot. The system may further include a computer comprising a processor configured to instruct a user to place a manufacturer's sizing guide on the distal cut surface for sizing the implant before the posterior cut surface is formed. The CAS device may assist in the formation of the distal cut surface and the posterior cut surface without having implant geometry data for the implant.

In accordance with another embodiment, an alignment pin assembly comprises an alignment pin and a sleeve configured to slide over a top portion of the alignment pin, wherein at least a portion of the alignment pin has a diameter (or width) that allows the posterior guide slot of a cut block to fit over/onto the alignment pin, thereby aligning the posterior guide slot with the planned location for forming the posterior cut surface, and thereby aligning the entire cut block on the bone in the planned POSE for forming the remaining cut surfaces on the bone.

In various alternative embodiments, the sleeve may include a coupling member (e.g., a square or hexagonal peg/nut) located at a proximal end of the sleeve, wherein the coupling member is configured to couple the alignment pin assembly to a CAS device (e.g., a 2-DoF device) by way of a coupling mechanism (e.g., a chuck, collet, interaction fit). A top portion of the alignment pin may be shaped to have a rectangular cross-section to fit inside an inner bore of the sleeve, where the inner bore is shaped to have a negatively matching rectangular cross-section to ensure that the alignment pin does not rotate inside the sleeve when the sleeve is driven and rotated to drive the alignment pin into a bone. The alignment pin may be a tiered alignment pin including a top tier (or top portion) and a bottom tier (or bottom portion), where the bottom tier has a diameter greater than a diameter of the top tier, and wherein the sleeve has an inner bore or opening having a diameter greater than the diameter of the top tier but less than the diameter of the bottom tier.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A depicts a femoral bone 10 and a femoral implant 12, where the femoral bone 10 was cut to form five cut surfaces including the anterior cut surface 14, the anterior chamfer cut surface 16, the distal cut surface 18, the posterior chamfer cut surface 20, and the posterior cut surface 22.

FIGS. 1B and 1C depict a femoral implant having a pair of pegs (23, 24) and five contact surfaces including the anterior contact surface 13, the anterior chamfer contact surface 15, the distal contact surface 17, the posterior chamfer contact surface 19, and the posterior contact surface 21.

FIG. 2 depicts a sizing guide mounted on the distal cut surface 18 formed on the femur 10.

FIGS. 3A and 3B depict a 4-in-1 cut block, where FIG. 3A is a top perspective view thereof, and FIG. 3B is a bottom perspective view thereof.

FIG. 4 depicts a graphical user interface (GUI) 30 operated by planning software for planning the location for forming cut surfaces on the bone to mount an implant in a desired POSE.

FIG. 5 depicts a GUI 31 for gap balancing the knee in the operating room.

FIG. 6 depicts a 2-DoF hand-held robot 100 (referred to hereinafter as 2-DoF device) for inserting pins (106a, 106b) in a bone ‘B’ coincident with a virtual plane ‘VP’.

FIG. 7 depicts a cutting guide 108 coupled to the pins (106a, 106b) inserted in the femur.

FIG. 8 depicts the 2-DoF device 100 maintaining alignment of the axis of a pin coincident with a second virtual plane ‘VP2’ for inserting pins (107a, 107b) in the distal cut surface 18.

FIG. 9 depicts the location for the second virtual plane “VP2” being defined such that when the cutting guide 108 is coupled to the pins (107a, 107b), the guide slot 112 is aligned with the predetermined location to form the posterior cut surface.

FIG. 10A depicts a spacer 120 for placement on the posterior cut surface to replace the removed posterior condyles in order to use a manufacturer's sizing guide in a conventional manner; FIG. 10B depicts a spacer 120′ having a non-adjustable thickness or fixed thickness; and FIG. 10C depicts the spacer 120 placed on the posterior cut surface 22 to replace the posterior condyles that were removed when forming the posterior cut surface.

FIG. 11 depicts a sizing guide mounted to the distal cut surface with the spacer 120 replacing the posterior condyles that were previously removed.

FIG. 12 depicts a 4-in-1 cut block 40 mounted to the distal cut surface according to the measurement from the sizing guide.

FIG. 13 depicts the femoral bone having all of the bone cuts from the above procedure.

FIGS. 14A and 14B depict an alignment guide for aligning a 4-in-1 cut block on the bone, where FIG. 14a is a front view thereof, and FIG. 14B is a perspective view thereof.

FIG. 15 depicts a distal cut surface and posterior cut surface formed on the bone and an alignment guide for placement on the posterior cut surface.

FIG. 16A depicts an alignment guide placed on a posterior cut surface formed on the bone and a 4-in-1 cut block for placement on a portion of the alignment guide.

FIG. 16B depicts a portion of an alignment guide placed inside the posterior guide slot of the 4-in-1 cut block to align the 4-in-1 cut block on the bone.

FIG. 17 depicts a digitizer digitizing surfaces of an implant to determine geometry data about the implant.

FIG. 18 depicts a digitizer digitizing guide slots of a cut block to determine geometry data about the cut block or geometry data about an implant associated with the cut block.

FIGS. 19A and 19B depict a first alignment pin assembly for aligning a cut block on a bone in a planned POSE, where FIG. 19A depicts the first alignment pin assembly in an assembled state, and FIG. 19B depicts the first alignment pin assembly in an exploded state.

FIGS. 20A and 20B depict a second alignment pin assembly for aligning a cut block on a bone in a planned POSE, where FIG. 20A depicts the first alignment pin assembly in an assembled state, and FIG. 20B depicts the first alignment pin assembly in an exploded state.

FIG. 21A depicts a CAS device used to insert a pin alignment assembly in the bone coincident with a virtual plane having a location corresponding to the location for forming a cut surface in the bone.

FIG. 21B depicts the alignment of a cut block on a bone by placing a guide slot of the cut block onto a portion of alignment pin assemblies inserted in the bone.

FIG. 22 is a schematic view showing the computer-assisted surgical system 100 including a 2-DoF device 102, a computing system 104, and a tracking system 106. In other inventive embodiments, the system includes an end effector 206 extending from a robotic arm.

FIG. 23A shows the 2-DOF device 102 in a first working POSE.

FIG. 23B illustrates the 2-DOF device 102 in a second working POSE.

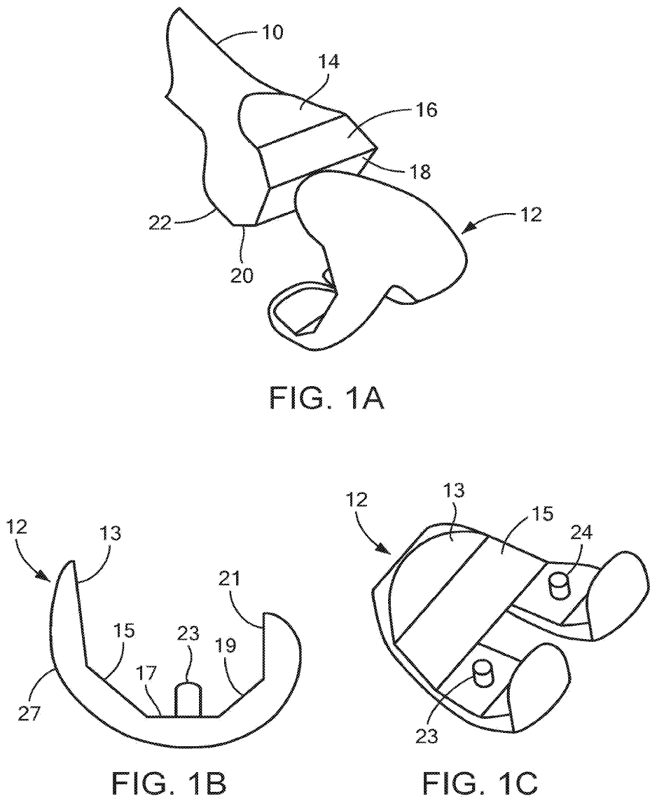

It should be noted that the foregoing figures and the elements depicted therein are not necessarily drawn to consistent scale or to any scale. Unless the context otherwise suggests, like elements are indicated by like numerals. The drawings are primarily for illustrative purposes and are not intended to limit the scope of the inventive subject matter described herein.

### DESCRIPTION OF ILLUSTRATIVE EMBODIMENTS

Definitions. As used in this description and the accompanying claims, the following terms shall have the meanings indicated, unless the context otherwise requires. Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references mentioned herein are incorporated by reference in their entirety.

The following description provides examples related to knee replacement; however, it should be appreciated that the embodiments described herein are readily adapted for use in a myriad of applications where it is desirous to position implants for joint replacement procedures in other portions of the body.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 1-4, 2-3, 2-4, and 3-4 and also may include factional values (e.g., 1.1-2, 1.2-2, etc.).

Unless indicated otherwise, explicitly or by context, the following terms are used herein as set forth below.

As used herein, like reference numerals described in with respect to subsequent drawings have the meaning imparted thereto with respect to the previously detailed drawings.

As used herein, the term “bone data” refers to data related to one or more bones. The bone data may be determined: (i) prior to making modifications (e.g., bone cuts, insertion of a pin or screw, etc.) to one or more bones, referred to as pre-operative bone data; and/or (ii) determined after one or more modifications have been made to a bone, referred to as post-modification bone data. The bone data may include: the shapes of the one or more bones; the sizes of the one or more bones; angles and axes associated with the one or more bones (e.g., epicondylar axis of the femoral epicondyles, longitudinal axis of the femur, the mechanical axis of the femur); angles and axes associated with two or more bones relative to one another (e.g., the mechanical axis of the knee); anatomical landmarks associated with the one or more bones (e.g., femoral head center, knee center, ankle center, tibial tuberosity, epicondyles, most distal portion of the femoral condyles, most proximal portion of the femoral condyles); bone density data; bone microarchitecture data; and stress/loading conditions of the bone(s). By way of example, the bone data may include one or more of the following: an image data set of one or more bones (e.g., an image data set acquired via fluoroscopy, computed tomography (CT), magnetic resonance imaging (MRI), ultrasound, other x-ray modalities, laser scan, etc.); three-dimensional (3-D) bone models, which may include a virtual generic 3-D model of the bone, a physical 3-D model of the bone, a virtual patient-specific 3-D model of the bone generated from an image data set of the bone; and a set of data collected directly on the bone intra-operatively commonly used with imageless CAS devices (e.g., laser scanning the bone, painting the bone with a digitizer). The term “virtual” may also be referred to herein as “digital”, meaning the data is stored, generated, and/or processed by a computer.

As used herein, the terms “computer-assisted surgical device” and “CAS device” refer to devices used in surgical procedures that are at least in part assisted by one or more computers. Examples of CAS devices illustratively include tracked/navigated instruments and surgical robots. Examples of a surgical robot illustratively include robotic hand-held devices, serial-chain robots, bone mounted robots, parallel robots, or master-slave robots, as described in U.S. Pat. Nos. 5,086,401; 6,757,582; 7,206,626; 8,876,830; 8,961,536; 9,707,043; and 11,457,980; which patents and patent application are incorporated herein by reference. The surgical robot may be active (e.g., automatic/autonomous control), semi-active (e.g., a combination of automatic and manual control), haptic (e.g., tactile, force, and/or auditory feedback), and/or provide power control (e.g., turning a robot or a part thereof on and off). It should be appreciated that the terms “robot” and “robotic” are used interchangeably herein. The terms “computer-assisted surgical system” and “CAS system” refer to a system comprising at least one CAS device and may further include additional computers, software, devices, or instruments. An example of a CAS system may include: i) a CAS device and software (e.g., cutting instructions, pre-operative bone data) used by the CAS device; ii) a CAS device and software (e.g., surgical planning software) used with a CAS device; iii) one or more CAS devices (e.g., a surgical robot); iv) a combination of i), ii), and iii); and iv) any of the aforementioned with additional devices or software (e.g., a tracking system, tracked/navigated instruments, tracking arrays, bone pins, rongeur, an oscillating saw, a rotary drill, manual cutting guides, manual cutting blocks, manual cutting jigs, etc.).

Also referenced herein is a “surgical plan.” A surgical plan is generated using planning software. The surgical plan may be generated pre-operatively, intra-operatively, or pre-operatively and then modified intra-operatively. The planning software may be used to plan the location for an implant with respect to a bone and/or plan a location to make one or more modifications (e.g., bone cuts, location for inserting bone pins) to the bone. The planning software may include various software tools and widgets for planning the surgical procedure. This may include, for example, planning: (i) a location for implant data (e.g., a 3-D implant model) with respect to bone data (e.g., a 3-D bone model) to define a location for the implant with respect to the bone; (ii) a location for one or more bone cuts to be made relative to bone data to define the locations for one or more cuts to be made on the bone, and/or (iii) one or more locations for inserting hardware (e.g., bone pins, screws) relative to bone data. All of which may be used to define locations for device operating data (e.g., a cut-file, a virtual plane, virtual boundary, a virtual axis) with respect to the bone data, where a CAS device is directed to control movement of an end-effector (e.g., the hardware, a burr, end-mill, drill bit) with respect to the bone according to the device operating data. The device operating data may also be referred to as data used for operating a device. The device operating data may also, or alternatively, be used to provide feedback (e.g., display instructions on a GUI) to a user for navigating one or more non-robotically controlled instruments (e.g., a tracked cutting tool, a tracked drill) relative to a bone.

As used herein, the term “digitizer” refers to a device capable of measuring, collecting, recording, and/or designating the position of physical locations (e.g., points, lines, planes, boundaries, etc.) in three-dimensional space. By way of example but not limitation, a “digitizer” may be: a “mechanical digitizer” having passive links and joints, such as the high-resolution electro-mechanical sensor arm described in U.S. Pat. No. 6,033,415 (which U.S. patent is hereby incorporated herein by reference); a non-mechanically tracked digitizer probe (e.g., optically tracked, electromagnetically tracked, acoustically tracked, and equivalents thereof) as described for example in U.S. Pat. No. 7,043,961 (which U.S. patent is hereby incorporated herein by reference); an end-effector of a robotic device; or a laser scanner.

As used herein, the term “digitizing” refers to the collecting, measuring, designating, and/or recording of physical locations in space using a digitizer. In some embodiments, “digitizing” may refer to the conversion of a designated location, area, or volume in space to a digital format. For example, a tracking system may determine the location of a digitizer probe tip in contact with a point on the bone, where the determined location of that point is saved to computer memory.

As used herein, the term “registration” refers to: the determination of the spatial relationship between two or more objects; the determining of a coordinate transformation between two or more coordinate systems associated with those objects; the mapping of an object onto another object; and a combination thereof. Examples of objects routinely registered in an operating room (OR) illustratively include: CAS systems/devices; anatomy (e.g., bone); bone data (e.g., 3-D virtual bone models); a surgical plan (e.g., location of virtual planes defined relative to bone data, cutting instructions defined relative to bone data, or other device operating data defined relative to bone data); and any external landmarks (e.g., a tracking array affixed to a bone, an anatomical landmark, a designated point/feature on a bone, etc.) associated with the bone (if such landmarks exist). Methods of registration known in the art are described in U.S. Pat. Nos. 6,033,415; 8,010,177; 8,036,441; and 8,287,522; and 10,537,388. In particular embodiments with orthopedic procedures, the registration procedure relies on the manual collection of several points (i.e., point-to-point, point-to-surface) on the bone using a tracked digitizer where the surgeon is prompted to collect several points on the bone that are readily mapped to corresponding points or surfaces on a 3-D bone model. The points collected from the surface of a bone with the digitizer may be matched using iterative closest point (ICP) algorithms to generate a transformation matrix. This transformation matrix and various other transformation matrices provides the mathematical locational relationship between: (i) bone data (e.g., a 3-D bone model, planned location for forming one or more cut surfaces; planned location for an implant model relative to a bone model); and/or a surgical plan (e.g., a pre-defined location for a targeted virtual plane that was defined with respect to bone data, a pre-defined location of device operating data that was defined with respect to bone data); and (ii) the coordinate system of a tracking array affixed to the bone (if present); or a CAS device (e.g., the base coordinate system of the CAS device, or a coordinate system of a tracking array affixed to the CAS device and, if needed, calibration data and/or kinematic data that define the location of an end-effector relative to the tracking array); and any other coordinate system or object required to perform the procedure. In other embodiments, the registration is performed using imageless registration.

As used herein, the term “display” is intended to encompass a variety of the digital devices that during operation provide an image (including multiple images in succession to form a video feed) recognizable to human viewing. Digital devices operative herein as displays illustratively include a graphical user interface (GUI), a computer or television (TV) monitor, a holographic display, a mobile display, a smartphone display, a video wall, a head-mounted display, a heads-up display, a virtual reality headset, a broadcast reference monitor, any of the aforementioned with a touchscreen capability, and a combination thereof. One or more computers comprising a processor may be operatively coupled to the display for controlling the output of the display.

As used herein, the term “feedback” may refer to visual feedback provided on a display. This “feedback” may also be provided in other forms, which may be in lieu of or in addition to visual feedback. For example, the “feedback” may include audio feedback, haptic/tactile feedback (e.g., a buzz or vibration when a digitizer tip is located at in an area of max deviation), or other visual feedback (e.g., a light on the surgical device may turn green or red depending on the amount of error between the surgical device and a planned cut surface).

Embodiments of the present invention provide a CAS system and method that allows the CAS system to be used with an unsupported implant. An unsupported implant is an implant in which the system lacks information about, or is not programmed with, the full geometry data of the implant. Thus, certain embodiments relate to an agnostic implant platform, which would allow the CAS device to be used for any implant line and/or implant manufacturer. One hurdle with an agnostic implant platform is the lack of implant geometry data from the implant manufacturers. The user/system is not equipped with 3-D implant models or the full implant geometry data to plan the location for forming all the cut surfaces on the bone (or generate a complete set of device operating data) and therefore a CAS device is unable to assist in forming all the bone cut surfaces. In addition, there may not be enough implant geometry data to determine an appropriately sized implant for the patient.

To overcome this lack of implant geometry data, embodiments of the CAS system may be configured to assist a user in planning locations for forming the distal cut surface and the posterior cut surface on the femur and the proximal cut surface on the tibia using at least one of: (i) the user's knowledge of the femoral implant distal thickness, the femoral implant posterior thickness, and the tibia implant thickness (which may include the tibia base plate thickness and may also include the various thicknesses of the tibia implant liners); (ii) publicly available information about the femoral implant distal thickness, the femoral implant posterior thickness, and the tibia implant thickness (which may include the tibia base plate thickness and also include the various thicknesses of the tibia implant liner); or (iii) via other means (e.g., a laser scan of the implant). A CAS device may then assist in forming the distal cut surface and posterior cut surface on the femur and the proximal cut surface on the tibia. The distal cut surface and posterior cut surface on the femur define how the femoral implant will mount on the bone in five degrees-of-freedom (anterior-posterior position, proximal-distal position, varus-valgus rotation, flexion-extension rotation, and internal-external rotation), which are the most relevant degrees-of-freedom that affect clinical outcomes. Therefore, certain embodiments can still leverage the accuracy of using a CAS system to assist in forming at least these two cut surfaces on the femur to accurately align the implant on the bone in five degrees-of-freedom without the full geometry data of the implant. Embodiments of the systems and methods described herein further assist the user in sizing the implant since there may not be enough implant geometry data available to the system or the user to determine an appropriately sized implant for the patient. Notching may occur if the implant is sized small and a poor implant fit may occur if the implant is sized large. Thus, in specific embodiments, systems and methods are described for overcoming this lack of implant geometry data in order to use a CAS device to assist in accurately forming at least two cut surfaces on the femur while further providing a method to determine an appropriately sized femoral implant for the patient.

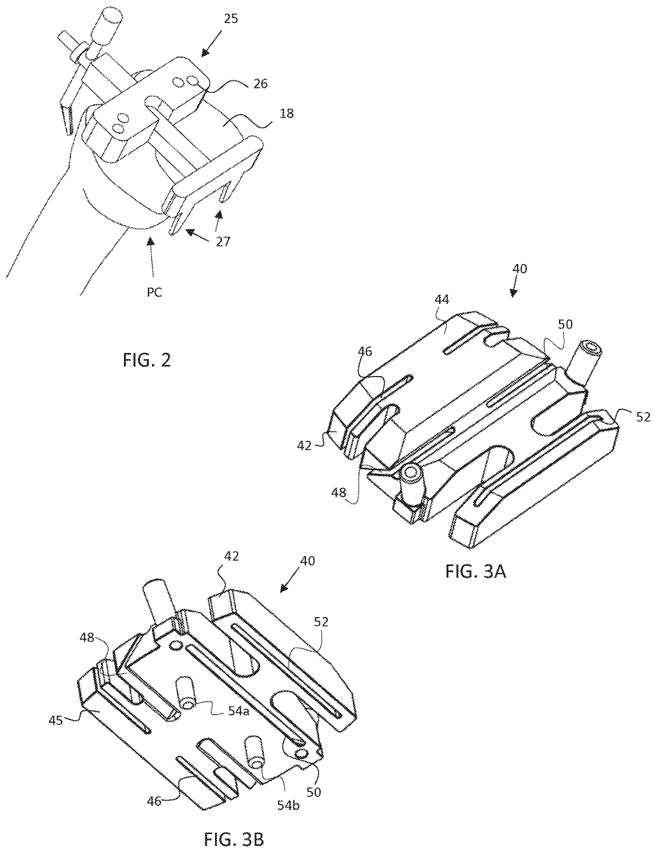

In particular embodiments, a CAS device may be used to assist in forming the femoral distal cut surface and the femoral posterior cut surface at a planned location on the bone. In doing so, the posterior condyles are removed, where those condyles are conventionally needed to mount the manufacture's sizing guide on the distal cut surface. For example, see FIG. 2. Therefore, in certain embodiments, after the posterior cut surface is formed, a spacer is placed on the posterior cut surface (directly thereon, or sandwiched between the posterior cut surface and the tibia plateau (or tibia cut surface)) to replace the removed posterior condyles in order to use the manufacturer's sizing guide to: (i) determine an appropriate implant size; and (ii) determine the location for mounting the 4-in-1 block on the distal cut surface to align the 4-in-1 cut block in the correct POSE to guide the formation of the remaining cut surfaces to accommodate the determined implant size. In some embodiments, the thickness of the spacer replicates (or matches) the thickness of the femoral implant posterior thickness, while in other embodiments, the thickness of the spacer replicates (or matches) the thickness of the removed posterior condyles.

The spacer may have an adjustment mechanism to account for the posterior thickness of different implant lines, or the thickness of the removed posterior condyles. For example, the thickness of the spacer can be adjusted between 8 and 11 millimeters (mm), which are common posterior thicknesses of femoral implants. In addition, or alternatively, multiple spacers of different thicknesses can be provided. The surgeon may then choose the correct sized spacer, or adjust the thickness of the spacer, in the OR that correlates to their chosen implant or matches the thickness of the removed posterior condyles. For example, the surgeon may know that a particular implant line has a femoral implant posterior thickness of 8 mm through their experience using the implants. The surgeon may plan to remove 8 mm from the posterior condyles when forming the posterior cut surface to match the posterior thickness of the implant. The surgeon may then place a spacer on the posterior cut surface that has a thickness of 8 mm to replicate the thickness of the posterior implant, which in this example is also the thickness of the removed posterior condyles. This allows the user to then mount the sizing guide onto the distal cut surface in a conventional manner for use to size the implant.

In another embodiment, a CAS device may be used to assist in the formation of the distal cut surface at a planned location on the femur. Then, a manufacturer's sizing guide is mounted onto the distal cut surface and used to determine an appropriately sized implant. After an implant size is determined, a CAS device is used to assist in the formation of the posterior cut surface at a planned location on the femur. An alignment device is then placed on the posterior cut surface for aligning the posterior guide slot of the 4-in-1 cut block with the location of the posterior cut surface as if the posterior guide slot of the 4-in-1 cut block was used to form the posterior cut surface. The 4-in-1 block is then secured to the distal cut surface at this location and used to guide a surgical saw blade in forming the remaining cut surfaces on the bone.

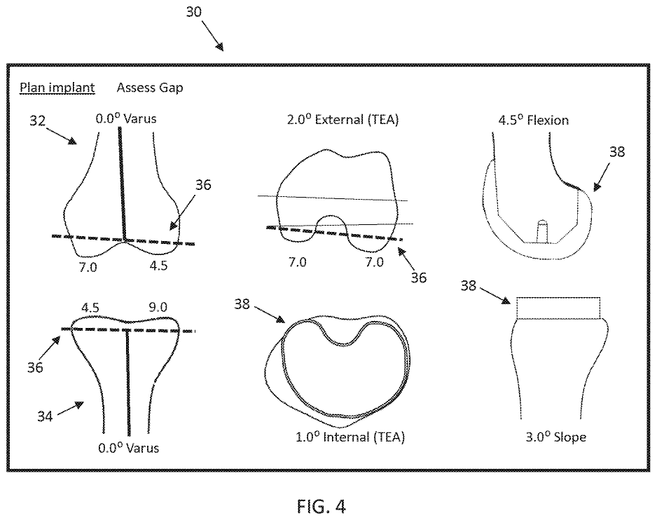

With reference now to FIG. 4, a graphical user interface (GUI) 30 is shown for planning a TKA procedure with an implant agnostic CAS system. The GUI 30 is operated by planning software for planning the location for forming cut surfaces on the bone to mount an implant in a desired POSE. The planning software may include models of the femur and tibia (also referred to as a femoral bone model 32 or tibial bone model 34), which may have been generated from an image data set (e.g., CT scan data) of the patient's bones. In a particular embodiment, the planning software may include two planning modes. The first planning mode may include a library of 3-D implant models that are supported by the system to allow the user to plan the POSE for an implant relative to a bone in a conventional pre-operative planning manner. The second planning mode (as shown in FIG. 4) is for a planning TKA procedures with an implant agnostic system (i.e., a system that lacks information, or is not programmed with, the full geometry data of the implant) by allowing the user (or system) to directly plan the location of the bone cuts for forming the cut surfaces on the bone. The planning software may include an option to switch between the first planning mode and the second planning mode. In other embodiments, the planning software is solely dedicated to planning TKA procedures for an implant agnostic system. For the implant agnostic planning software, instead of displaying and moving a 3-D implant model relative to the 3-D bone models, the GUI 30 displays a graphic 36 (e.g., line, box, grayed out bone areas) showing the location of the planned bone cuts. The system may also show a generic implant model 38 as a visualization tool to assist in planning the location for the implant relative to the bone. The GUI 30 may also display alignment information to assist in planning the alignment of the implant on the bone in relevant clinical degrees-of-freedom. This information may include the degrees of varus-valgus of the bone with respect to the mechanical axis as a result of a planned distal cut, the degrees of external-internal rotation of the implant from a bone condylar axis (e.g., transepicondylar axis (TEA), posterior condylar axis (PCA)) as a result of the planned posterior cut, the degrees of flexion-extension of the implant with respect to the bone as a result of the planned distal cut and/or planned posterior cut, the tibial posterior slope of the implant with respect to the bone as a result of the planned tibial cut, etc. The GUI may also display an amount of bone that will be resected for a given bone cut (e.g., 7.0 mm on the lateral distal condyle and 4.5 mm on the medial distal condyle as shown in the top left window of the GUI 30 of FIG. 4). The planned location for forming the cut surfaces relative to the bone data (e.g., 3-D bone model) is then saved for future use (e.g., for use by the CAS device) once the user is satisfied with the plan. The planned location of any device operating data (e.g., the planned location of a virtual plane or virtual boundary) relative to the bone data may also be saved for future use. In the OR, the bone is exposed and registered to the bone data (e.g., bone model) using registration techniques known in the art, which allows the system to determine the planned locations for forming the cut surfaces relative to the real-time location of the bone and/or determine the location of any device operating data (e.g., the location of a virtual plane) relative to the location of the bone.

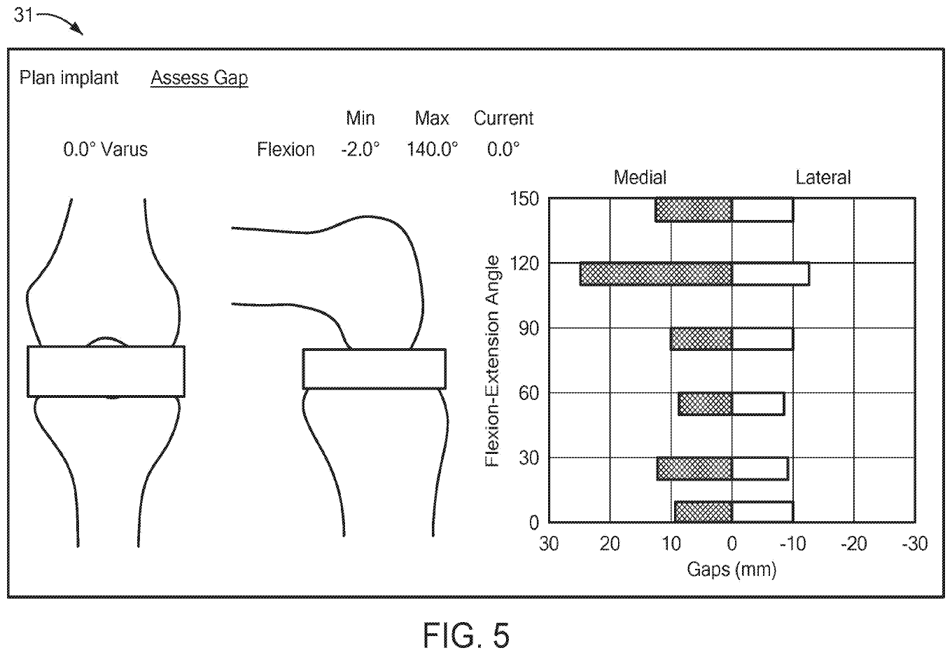

FIG. 5 depicts a GUI 31 for gap balancing the knee in the operating room. Before gap balancing, a first tracking array is affixed to the femur and a second tracking array is affixed to the tibia. The bone data is then registered to the femur and tibia (and more specifically to the coordinate system of the tracking arrays affixed to each bone, respectively) using techniques known in the art. For example, the 3-D bone models of the femur and tibia may be registered to the locations of each bone, respectively, by matching points digitized on the bone with corresponding points/surfaces on the 3-D bone models.. A tracking system may then track the location and movement of the femur and tibia by way of the tracking arrays affixed thereto and the system can determine the corresponding location and motion of the 3-D bone models using the registration data (i.e., the registered location of each 3-D bone model to the coordinate system of each tracking array, respectively). The system can also determine the planned location for the bone cuts and/or the device operating data relative to the real-time location of each bone, respectively, using the registration data and the surgical plan. To perform gap balancing, the user applies tension to the surrounding ligaments by either placing a tensioner between the two bones or by pulling the tibia away from the femur (e.g., in varus-valgus, or proximal-distal) to force tension on the ligaments. While applying tension on the ligaments, the user flexes and extends the knee throughout the knee's range of motion (e.g., 0 degrees is fully extended and as much as the knee can flex >0 degrees). [Note, gap balancing may occur before any bone cuts are made, or in some case, the user may choose to make the tibia bone cut first and then perform gap balancing.] The tracking system records the movement of the bones, via the tracking arrays, and determines the corresponding motion of the registered bone models throughout this motion. In some embodiments, the GUI 31 may display the 3-D bone models and movement of the 3-D bone models corresponding to the real-time movement of the bones. A computer operatively coupled to the tracking system determines the medial and lateral gaps between the bones at two or more flexion-extension locations (one with the knee fully extended and another with the knee fully flexed). The computer may determine the gaps by measuring the distance between the distal portion of the femoral bone model (e.g., a point on the outer surface of the femoral bone model that is closest to the tibia bone model) and the proximal portion of the tibial bone model (e.g., a point on the outer surface of the tibia bone model) for each angle throughout this flexion-extension motion. The graph on the right shows the gaps at six different flexion-extension locations (or angles), where the pattern filled bars are the medial gap measurements and the un-filled bars represent the lateral gap measurements. One of the goals of gap balancing is to obtain an equal gap (medial gap=lateral gap) in both flexion and extension. If the gaps aren't balanced, the user may choose to adjust the planned location for a bone cut. For example, the user may rotate the implant internally or externally to obtain an equal medial-lateral gap in flexion, and varus or valgus to obtain an equal medial-lateral gap in extension. The user may also move the implant distally or proximally to decrease or increase the size of the gap in extension, or posterior or anterior to decrease or increase the size of the gap in flexion, to make sure there is enough room for the implants to fit in the knee and that the ligaments will be properly tensioned when the implants are positioned in the knee. The bone models shown on the left of the GUI 31 shows equal gaps in extension and the bone models shown in the middle of the GUI 31 shows equal gaps in flexion. The new or updated locations for forming the cut surfaces on the bone is then saved for use by the CAS device as further described below.

It should be appreciated that while FIG. 5 shows the gaps being measured from the distal portion of the femoral bone model to the proximal portion of the tibia bone model, embodiments of the present invention may also include different points for measuring the gap distances. For example, the gaps may be measured between: (i) one or more points on a surface of a femoral implant at a planned location on the femoral bone model and one or more points on a planned location for forming a cut surface on the tibia bone model; (ii) one or more points on the surface of the tibial implant model and one or more points on the surface of the femoral implant model; (iii) one or more points on a planned location for any other cut surface on a bone model and one or more points on an implant model; or (iv) any other combination of measurements between points on an implant model, a bone model, or a planned location of a cut surface. The points for measuring the gap distances may also include points located on cut surfaces formed on the bone (e.g., a point on the femoral distal cut surface 18 formed on the femur ‘F’, or a point on the proximal tibia cut surface formed on the tibia ‘T’).

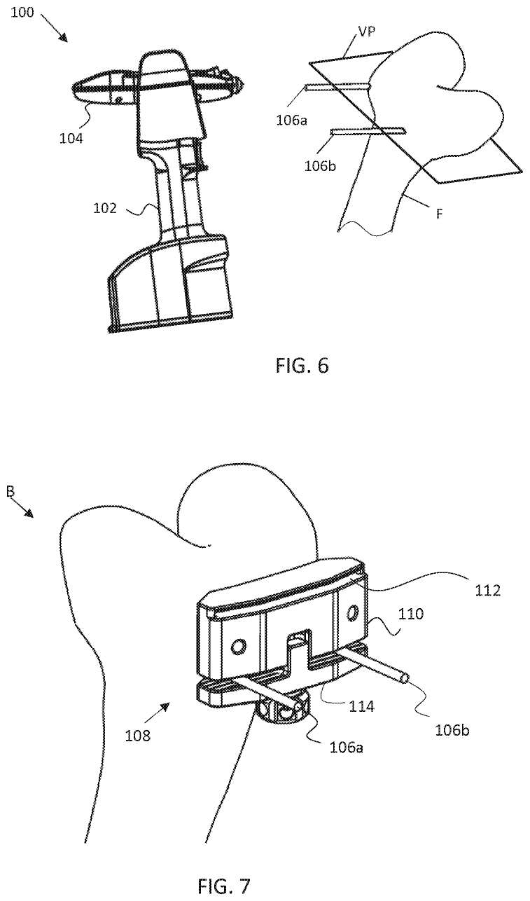

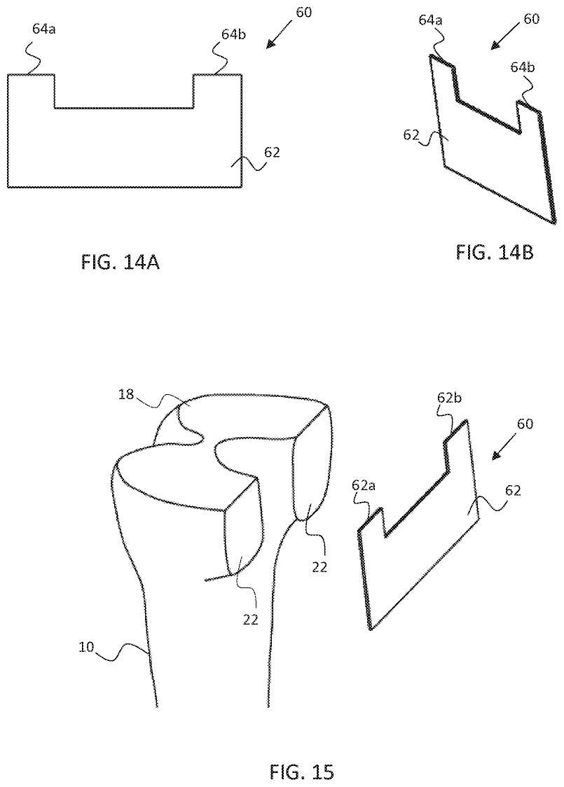

FIGS. 6-9 depict an implant agnostic system and method for forming the distal femoral cut surface and the posterior femoral cut surface at the planned (or pre-determined) locations using a 2-DoF hand-held surgical robot as further described below with reference to FIGS. 13-14B . However, the distal cut surface and posterior cut surface may be formed with other CAS devices, including the system and method described in PCT Publication No. WO/2023/059931 for PCT Application No. PCT/US2022/046146, which is hereby incorporated herein by reference in its entirety.

FIG. 6 depicts a 2-DoF hand-held robot 100 (referred to hereinafter as 2-DoF device) for inserting pins (106a, 106b) in a femur ‘F’ coincident with a virtual plane ‘VP’. The 2-DoF device 100 includes a hand-held portion 102, a working portion 104, and actuators housed in the hand-held portion 102 that receive control signals to maintain alignment of an axis of a pin, coupled to the working portion 104, coincident with the virtual plane “VP”. The location for the virtual plane “VP” is defined relative to the femoral bone model based on the pre-determined location for forming the distal cut surface and geometry data of a cutting guide 108 shown in FIG. 7 as further described below. The 2-DoF device 100 is configured to be held and fully supported by the user. While the user manually advances the 2-DoF device 100 towards the femur ‘F’, the actuators receive control signals from a computer/controller to maintain the alignment of the pin axis coincident with the virtual plane “VP” to account for the surgeon's hand movements (and bone movements) to ensure the pin is inserted in the bone coincident with the virtual plane “VP”. Generally, at least two pins (106a, 106b) are inserted in the femur ‘F’in this manner.

A cutting guide 108 is then coupled to the pins (106a, 106b) inserted in the femur ‘F’ as shown in FIG. 7. The cutting guide 108 includes a body 110 having a guide slot 112 and a coupling portion 114. The guide slot 112 is configured to receive a cutting tool (e.g., surgical saw blade) therethrough, where a user advances the cutting tool through the guide slot 112 to form the distal cut surface on the femur ‘F’. The coupling portion 114 couples the body 110 to the pins (106a, 106b). A clamping mechanism may cause the coupling portion 114 to move relative to the body 110 to clamp the cutting guide 108 onto the pins (106a, 106b). The location of the virtual plane ‘VP’ is defined such that when the cutting guide 108 is coupled to the pins (106a, 106b), the guide slot 112 aligns with the pre-determined location for forming the distal cut surface 18. Essentially, the location for the virtual plane ‘VP’ is defined as a plane offset from the pre-determined location for the distal cut surface by the known distance between the guide slot 112 and the bottom surface of the body 110 plus a half-width (or radius) of a pin. Once the cutting guide 108 is coupled to the pins (106a, 106b), a user may advance a surgical saw blade through the guide slot 112 to form the distal cut surface 18.

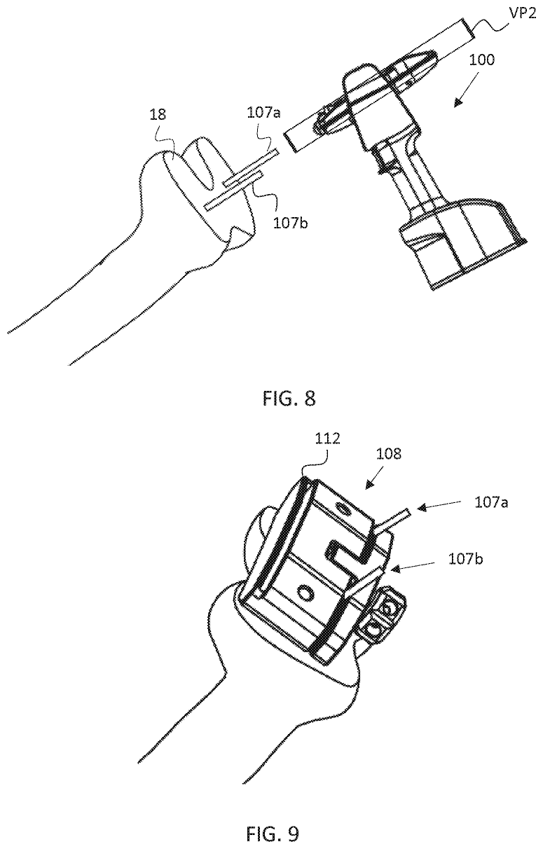

FIG. 8 depicts the 2-DoF device 100 maintaining alignment of the axis of a pin coincident with a second virtual plane ‘VP2’ for inserting pins (107a, 107b) in the distal cut surface 18. The location for the second virtual plane “VP2” is defined such that when the cutting guide 108 is coupled to the pins (107a, 107b) (as shown in FIG. 9), the guide slot 112 is aligned with the pre-determined location to form the posterior cut surface 22 as shown in FIG. 9. Essentially, the location for the second virtual plane ‘VP2’ is defined as a plane offset from the pre-determined location for the posterior cut surface by the known distance between the guide slot 112 and the bottom surface of the body 101 plus a half-width (or radius) of a pin. Once the cutting guide 108 is coupled to the pins (107a, 107b), a user may advance a surgical saw blade through the guide slot 112 to form the posterior cut surface 22.

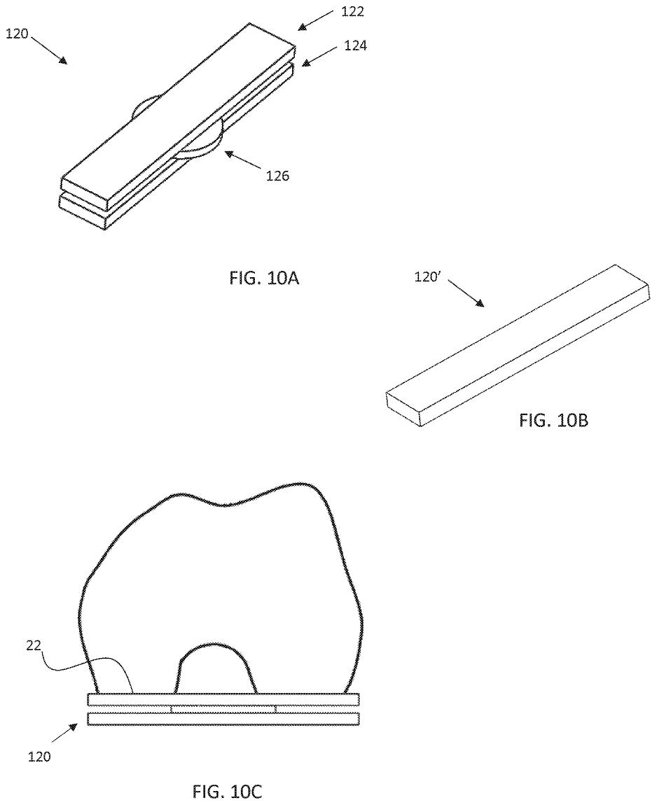

FIG. 10A depicts a spacer 120 for placement on the posterior cut surface to replace the removed posterior condyles in order to use a manufacturer's sizing guide in a conventional manner. The thickness of the spacer 120 may be adjustable or selectable to match the thickness of the bone that was resected from the posterior condyles, or to match the posterior thickness of the implant. The spacer 120 may include a top platform 122, a bottom platform 124, and an adjustment mechanism 126. The thickness of the spacer 120 may be adjustable via the adjustment mechanism 126, which when operated (e.g., rotate a knob or wheel, push a button, etc.) moves the top platform 122 and bottom platform 124 either towards or away from one another thereby changing the thickness of the spacer 120. Without limitation, some example thickness adjusting mechanisms include a rotary knob connected to a cam, a linear stage (e.g., as described in https://en.wikipedia.org/wiki/Linear_stage, the contents of which are hereby incorporated by reference), a knob adjust variable height platform (e.g., as described in https://www.newport.com/f/knob-adjust-variable-height-platform, the contents of which are hereby incorporated by reference), or other thickness adjusting mechanism. The spacer may also include notches or numbers to show the user the current thickness of the spacer (e.g., 8 mm, 10 mm, 11 mm, etc.). For example, if a surgeon prefers a particular implant line with implants having a posterior thickness of 8 mm, then the adjustable spacer may be set to ‘8’. In certain embodiments, the adjustment mechanism 126 may allow the spacer to be adjusted in 0.5 mm increments with an adjustable range between 8 mm and 11 mm. In other embodiments, multiple spacers of different thicknesses may be provided in the operating room (OR) and the surgeon can select which spacer to use, or the spacers may be configured to be stackable in order to provide spacers of different thicknesses. For example, FIG. 10B depicts a spacer 120′ having a non-adjustable thickness or fixed thickness, where multiple spacers 120′ of different thicknesses may be provided in the OR for selection by the user. FIG. 10C depicts the spacer 120 placed on the posterior cut surface 22 to replace the posterior condyles that were removed when forming the posterior cut surface. In some embodiments, the system may instruct the user to place the spacer (120, 120′) on the posterior cut surface via a graphical user interface GUI located in the OR, or via other feedback mechanisms (e.g., an instruction manual).

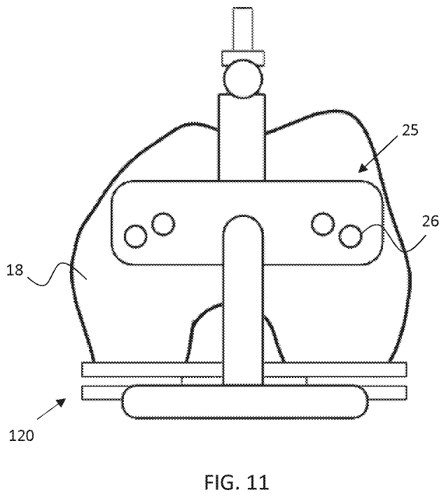

FIG. 11 depicts a sizing guide 25 mounted to the distal cut surface with the spacer 120 replacing the posterior condyles that were previously removed. The sizing guide 25 is generally provided by the implant manufacturer and is specific to the manufacturer's implant line. These sizing guides 25 include paddles 27 (shown in FIG. 2) or other posterior condyle referencing features that reference (e.g., abut against) the uncut posterior condyles in order for the sizing guide to be used according to the manufacturer's specifications. Since the posterior condyles have been cut with the accuracy of a CAS device, the spacer 120 may be placed on the posterior cut surface to replace the removed posterior condyles to use the sizing guide 25 as manufactured (e.g., the paddles 27 or other posterior condyle referencing feature of the sizing guide 25 may now reference the bottom surface of the spacer 25). The system may then instruct the user to mount the sizing guide 25 on the distal cut surface via a graphical user interface GUI located in the OR, or via other feedback mechanisms (e.g., an instruction manual). With the sizing guide 25 mounted to the distal cut surface 18, the sizing guide 25 may be set to 0 degrees of rotation from the posterior condyle axis and the user may then determine an appropriately sized implant with the sizing guide 25. Once the implant size is determined, the guide holes 26 in the sizing guide 25 are aligned for a surgeon to insert pins or drill peg holes in the bone, where the inserted pins or drilled peg holes are used to align a 4-in-1 cut block in the correct POSE for forming the remaining bone cuts on the bone.

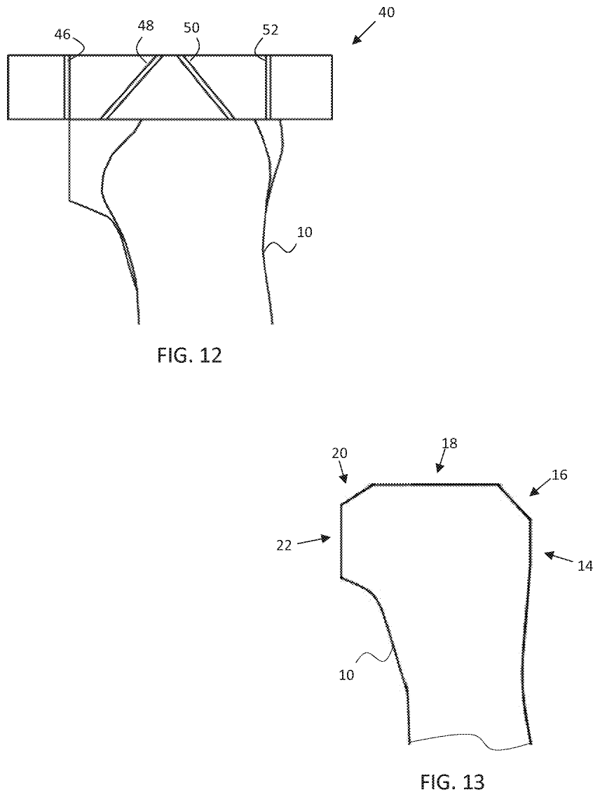

FIG. 12 depicts a 4-in-1 cut block 40 mounted to the distal cut surface according to the measurement from the sizing guide 25. The 4-in-1 cut block 40 in some embodiments includes an anterior guide slot 52, an anterior chamfer guide slot 50, a posterior chamfer guide slot 48, and a posterior guide slot 46, although other configurations may be provided in alternative embodiments. The 4-in-1 cut block 40 may further include a set of pegs (e.g., a pair of pegs) that fit into the peg holes drilled in the bone with the aid of the guide holes 26 in the sizing guide 25. A user may then advance a surgical saw blade through each of the guide slots to form the remaining cut surfaces on the bone.

FIG. 13 depicts the femoral bone having all of the bone cuts from the above procedure.

Returning back to FIGS. 10A to 11, in particular embodiments, the thickness of the spacer (120, 120′) is configured to replicate (or match) the posterior thickness of the femoral implant as previously described. This provides the surgeon with more flexibility when planning the location for the posterior cut surface that will still allow the use of the manufacturer's sizing guide. In certain situations, the user may choose to resect the posterior femur (e.g., the posterior condyles) by more or less than the posterior thickness of the implant. For example, the surgeon while assessing the soft tissues of the knee (e.g., using the gap balancing technique as described above) may determine that the posterior cut surface should be formed more anteriorly or posteriorly on the bone at a location that results in a resection amount that does not equate to the posterior thickness of the implant. In conventional TKA with manual instruments, the posterior cut surface is typically formed at a location that results in a resection amount corresponding to the posterior thickness of the implant. For example, if the posterior thickness of the implant is 8 mm, then the posterior resection amount is also 8 mm. This is to maintain the joint line when the implant components are installed in the knee. However, computer-assisted surgery gives the surgeon more tools to position the implant components according to their surgical philosophies and/or preferences. One such tool is the planning software that allows the surgeon to assess the bones and then position the implant models relative to the bone models to designate the best fit and alignment. CAS systems may also allow the surgeon to assess the soft tissues surrounding the knee, such as the gap balancing technique described above, and then make adjustments to the planned locations for forming the cut surfaces to ensure, among other things, that the ligaments are properly tensioned when the implants are installed in the knee. Thus, with these advanced tools, the location for forming the posterior cut surface on the bone may result in a resection amount that does not match the posterior thickness of the femoral implant. It also allows the surgeon to determine a desired internal-external rotation for the femoral implant, which may result in different resection amounts between the medial posterior condyle and the lateral posterior condyle.

With all this in consideration, there is one factor that needs to be addressed when using the manufacturer's sizing guide, and that is the fact that the manufacturer's sizing guides have specific dimensions that ensures the resulting posterior resection amount on the femur will always equal the posterior thickness of the implant. In a conventional TKA procedure, the user places the sizing guide 25 on the distal guide surface to determine an appropriately sized implant and I-E rotation for the implant. Once a desired implant size and I-E rotation is determined, the guide holes 26 of the manufacturer's sizing guide 25 are used to guide a drill bit in forming the peg holes in the distal cut surface. The sizing guide is specifically dimensioned, such that when the pegs of the 4-in-1 cut block are then placed in those formed peg holes, the posterior guide slot 46 of the 4-in-1 block 40 is aligned on the bone at a location that will always result in a posterior resection amount equal to the posterior thickness of the implant. This is to preserve the joint line of the knee when using manual instrumentation. However, with the aforementioned tools (e.g., planning, gap balancing) of a CAS system, the surgeon has more discretion on where to plan the formation of the posterior cut surface, which may not result in a posterior resection amount equal to the posterior thickness of the femoral implant. To address this issue, a spacer (120, 120′) having a thickness that replicates (or matches) the posterior thickness of the femoral implant is used. This will place the guide holes 26 of the manufacturer's sizing guide 25 in the correct location for forming the peg holes and sizing the implant as if the posterior resection amount was equal to the posterior thickness of the implant regardless of the actual resection amount. This is a work around that allows the manufacturer's sizing guide to be used to size the implant and position the guide holes 26 in the correct POSE for aligning the 4-in-1 block when the surgeon plans for a posterior resection amount that is not equal to the posterior thickness of the implant. For example, consider a femoral implant having a posterior thickness of 8 mm. After gap balancing, the surgeon decides to form the posterior cut surface at a location on the bone that removes 6 mm of bone (i.e., a posterior resection amount of 6 mm). The surgeon then mistakenly places a spacer having a thickness equal to the thickness of the removed condyles (i.e., a spacer having a thickness of 6 mm) on the posterior cut surface to size the implant. After sizing, the surgeon advances a drill bit through the guide holes 26 to form the peg holes. In this case, when the pegs (54a, 54b) of the 4-in-1 block 40 are placed in the peg holes, the posterior guide slot 46 of the 4-in-1 cut block 40 is aligned relative to the bone at a location that would result in an 8 mm posterior resection amount, which would be a 2 mm “over-resection” from the actual posterior cut surface formed at 6 mm. Thus, all the guide slots of the 4-in-1 block would be shifted anteriorly by 2 mm. To account for this “over-resection”, a spacer having a thickness of the implant (8 mm) may be placed on the posterior cut surface to size the implant with the sizing guide 25 (instead of a using a spacer of 6 mm). After sizing, the surgeon advances a drill bit through the guide holes 26 to form the peg holes. In this case, when the pegs (54a, 54b) of the 4-in-1 block 40 are placed in the peg holes, the posterior guide slot 46 of the 4-in-1 cut block 40 is aligned relative to the bone at a location that would result in a 6 mm posterior resection amount, which aligns with the actual posterior cut surface formed at 6 mm. All the guide slots now align in the correct POSE for forming the remaining cut surfaces on the bone. In another example, using the same implant, the surgeon may decide to form the posterior cut surface with a posterior resection amount of 10 mm. The surgeon then mistakenly places a spacer having a thickness equal to the thickness of the removed condyles (i.e., a spacer having a thickness of 10 mm) on the posterior cut surface to size the implant. After sizing, the surgeon advances a drill bit through the guide holes 26 to form the peg holes. In this case, when the pegs (54a, 54b) of the 4-in-1 block 40 are placed in the peg holes, the posterior guide slot 46 of the 4-in-1 cut block 40 is aligned relative to the bone at a location that would result in an 8 mm posterior resection amount, which would be a 2 mm “under-resection” from the actual posterior cut surface formed at 10 mm. Thus, all the guide slots of the 4-in-1 block would be shifted posteriorly by 2 mm. To account for this “under-resection”, a spacer having a thickness of the implant (8 mm) may be placed on the posterior cut surface to size the implant with the sizing guide 25 (instead of a using a spacer of 10 mm). After sizing, the surgeon advances a drill bit through the guide holes 26 to form the peg holes. In this case, when the pegs (54a, 54b) of the 4-in-1 block 40 are placed in the peg holes, the posterior guide slot 46 of the 4-in-1 cut block 40 is aligned relative to the bone at a location that would result in a 10 mm posterior resection amount, which aligns with the actual posterior cut surface formed at 10 mm. All the guide slots now align in the correct POSE for forming the remaining cut surfaces on the bone.

In addition, in a conventional TKA procedure, the manufacturer's sizing guides have adjustment mechanisms that allow the user to set the desired internal-external (I-E) rotation for the femoral implant (e.g., 3° external to the posterior condylar axis). However, using the CAS tools described herein (e.g., planning software, gap balancing), the user has more options to determine or plan the location for the posterior cut surface, which ultimately defines the I-E rotation for the femoral implant. Therefore, when the user is using the manufacturer sizing guide with the spacer (120, 120′) having a thickness equal to the posterior thickness of the implant, the user can set the I-E adjustment mechanism on the sizing guide to 0° of rotation from the posterior condylar axis, which will maintain the I-E rotation as defined by the posterior cut surface. In other words, with the adjustment mechanism of the sizing guide on this setting (0° of rotation from the posterior condylar), the sizing guide will not change the I-E rotation as defined by the posterior cut surface.

In other embodiments, the thickness of the spacer (120, 120′) replicates, or matches, the thickness of the removed posterior condyles. In which case, the manufacturing sizing guide can be used with a spacer (120, 120′) having a thickness equal to the thickness of the removed posterior condyles.

It should further be appreciated that some manufacturer's sizing guides may be specifically dimensioned for referencing the anterior face of the femur. Up until now, the manufacturer's sizing guide have been described as posterior referencing, where the sizing guide is specifically dimensioned to reference the posterior condyles for sizing the implant and aligning the posterior guide slot of the 4-in-1 guide that results a posterior resection amount equal to the posterior thickness of the implant. For some manufacturer's sizing guides, the sizing guides are dimensioned to reference the anterior face of the femur for sizing the implant and aligning the anterior guide slot of the 4-in-1 guide that results in an anterior resection amount equal to the anterior thickness of the implant. Thus, in some embodiments, the user may plan the location for forming the distal cut surface and the anterior cut surface and then use a CAS device to assist in forming the distal cut surface and the anterior cut surface. In this case, a spacer (120, 120′) having a thickness equal to the anterior thickness of the implant may be placed on the anterior cut surface for using an anterior referencing manufacturer's sizing guide to size the implant and determine the location for forming the peg holes to align the 4-in-1 block on the distal cut surface. The anterior cut surface also defines the I-E rotation for the femoral implant, so the I-E adjustment setting on the sizing guide is set to 0° of rotation from the posterior condylar axis to maintain the desired I-E rotation of the femoral implant as defined by the anterior cut surface. It's possible that this may be applied to the distal cut surface and any other cut surface on the femur. Thus, in some embodiments, a user may use a CAS system to plan and form the distal cut surface and at least one other cut surface on the femur then use a spacer and the manufacturer's sizing guide in the same manner as previously described for that at least one other cut surface on the femur.

With reference now to FIGS. 14A-16B , a specific embodiment for using a CAS device to assist in forming at least two cut surfaces on the femur while further providing a method to determine an appropriately sized femoral implant for the patient is shown. The same system and method may be used to plan the locations and form the femoral distal cut surface and the femoral posterior cut surface as described above with reference to FIGS. 4 to 7. Then after the distal cut surface 18 is formed, the manufacturer's sizing guide 25 is placed on the distal cut surface 18 and used to size the implant, where the paddles 27 of the sizing guide 25 can reference the posterior condyles as shown in FIG. 2. In this embodiment, the sizing guide 25 is used before the posterior cut surface is formed on the femur for sizing the implant. After the user determines an appropriately sized implant for the patient, the sizing guide 25 is removed and the posterior cut surface 22 is formed in the same manner as described with reference to FIGS. 8 and 9. Briefly, a 2-DoF device 100 maintains alignment of a pin 107 coincident with a second virtual plane “VP2” for inserting pins (107a, 107b) in the distal cut surface 18 coincident with the second virtual plane “VP2”. A cut guide 108 is then coupled to the pins, and a user advances a surgical saw blade through the guide slot 112 to form the posterior cut surface 22. The location of the second virtual plane “VP2” is defined relative to the registered bone model such that the guide slot 112 of the cut guide 108 will align with the planned location for forming the posterior cut surface 22.

The next step is to align the 4-in-1 cut block 40 on the distal cut surface 18. FIGS. 14A and 14B depict an alignment guide 60 for aligning a 4-in-1 cut block 40 in the correct POSE relative to the bone to guide the formation of the remaining cut surfaces on the femur. The alignment guide 60 includes a body 62 and one or more alignment features (62a, 62b). The one or more alignment features (62a, 62b) are configured to fit inside the posterior guide slot 46 of the 4-in-1 cut block 40 to align the posterior guide slot 46 with the posterior cut surface 22 formed on the bone. In doing so, the other guide slots of the 4-in-1 cut block 40 will align on the bone in the correct POSE to guide the formation of the remaining cut surfaces. The thickness of the one or more alignment features (62a, 62b) may therefore be less than the size of the opening of the posterior guide slot 46. In some embodiments, the thickness of the alignment features (62a, 62b) ranges from 0.5 mm to 1.5 mm in order to fit inside (or slide into) the opening of the posterior guide slot 46. In some embodiments, the body 62 of the alignment guide 60 has the same thickness as the alignment features (62a, 62b), while in other embodiments, the body 62 may have a different thickness than the alignment features (62a, 62b). The alignment features (64a, 64b) may be prongs, projections, or extensions, that extend from the body 62 of the alignment guide 60, while in other embodiments, the alignment features (64a, 64b) are part of the body 62 that does not necessarily extend from any portion of the body 62 (e.g., a portion or area of the body 62 may fit inside the posterior guide slot 46). The body 60 may include at least one face configured for placement against the posterior cut surface 62. For example, FIG. 15 depicts the alignment guide 60 right before it is placed against the posterior cut surface 22. The alignment guide 50 may be made of any suitable material (e.g., metals, plastics, etc.), and may be single use or sterilizable for multiple uses. The alignment guide 60 may further include a handle (not shown) for easy handling by the user.

In some embodiments, the alignment guide 60 is a separate device as part of a surgical kit for use during the procedure. In other embodiments, the alignment guide 60 is a device commonly used during a TKA procedure or any other surgical procedure for that matter. For example, the alignment guide 60 may be a surgical saw blade readily available in the operating room (OR), where a face of the surgical saw blade is placed against the posterior cut surface 22 and then the posterior guide slot 46 slides down onto a tip of the saw blade. Other devices readily available in the OR that may be used as an alignment guide 60 include a retractor, pins, clamps, and other surgical hardware. As such, it should be appreciated that many devices and/or configurations of devices may be used as the alignment guide 60 as long as the device is capable of referencing the location of the posterior cut surface 22 (e.g., placing a face of the device against the posterior cut surface) and include some portion that can reference (e.g., fits inside) the posterior guide slot 46 in order to align the posterior guide slot 46 with the location of the posterior cut surface 22.

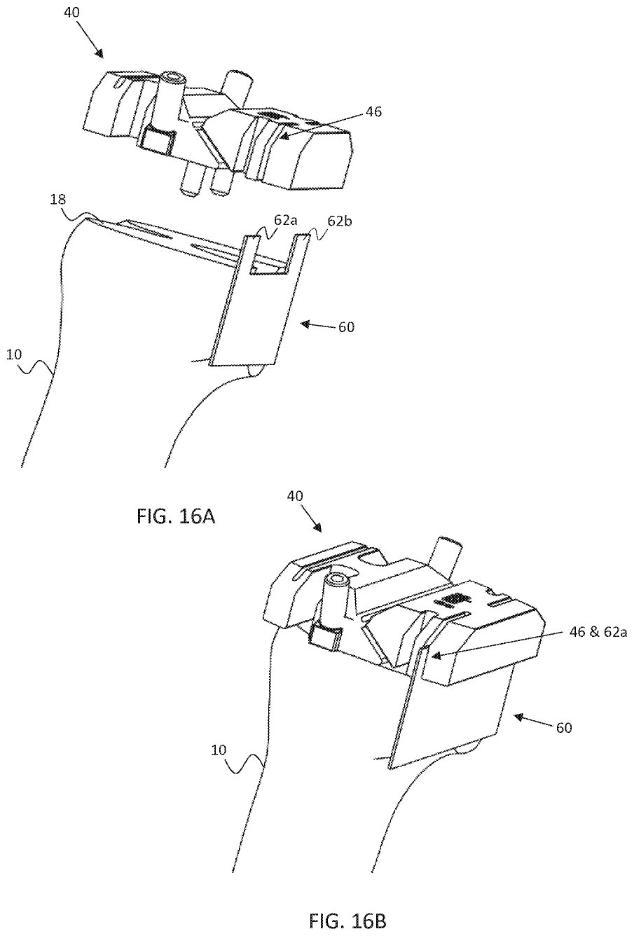

With reference to FIGS. 16A and 16B, the alignment guide 60 is shown placed against the posterior cut surfaces (22a, 22b). The alignment guide 60 may be held in place by the user, while in other embodiments, a clamp or other fixation device may be used to hold the alignment guide 60 against the posterior cut surface 22. In one embodiment, the alignment guide 60 includes one or more spikes, prongs, or other bone interacting elements (not shown) extending from a face of the body 62 to interact with the bone to hold the alignment guide 60 to the posterior cut surface 22. After the alignment guide 60 is placed on the posterior cut surface 22 as shown in FIG. 16A, the posterior guide slot 46 of the 4-in-1 cut block 40 is then placed on the alignment features (62a, 62b) as shown in FIG. 16B. FIG. 16B depicts the 4-in-1 cut block 40 positioned on the distal cut surface 18, where the alignment features (62a, 62b) are positioned inside the posterior guide slot 46 to align the posterior guide slot 46 with the posterior cut surface 22. The user may hammer the 4-in-1 cut block 40 into the distal cut surface 18 to secure the pegs (54a, 54b) of the cut block 40 in the bone. The use may in addition, or alternatively, place pins or screws through designated areas in the cut block 40 and into the bone to secure the 4-in-1 cut block at the aligned location. After the 4-in-1 cut block 40 is secured to the distal cut surface 18, the user may advance a surgical saw blade through the other guide slots to form the remaining cut surfaces on the bone. The femoral implant is then installed on the cut surfaces to complete the femoral side of the TKA procedure.

The above procedure allows the use of a manufacturer's sizing guide 25 to size the femoral implant while maintaining the desired/planned location for forming the posterior cut surface 22. In some situations, the planned location for the posterior cut surface 22 may result in too much internal-external rotation for the determined size for the implant, which may result in notching. To prevent this notching, the user may simply increase the size of the implant and form the cut surfaces with a cut block having dimensions corresponding to the new implant size.

### Determining Implant Dimensions

Embodiments of the present invention also provides a system and method for determining geometry data about an implant and/or cut block in order to use an unsupported implant with a CAS system. The system and method utilizes a digitizer to digitize portions of the implant or portions of the cut block to determine relevant geometry data (or dimensions) of the implant and/or cut block that can then be used to plan and/or execute a TKA procedure with that implant or implant line.

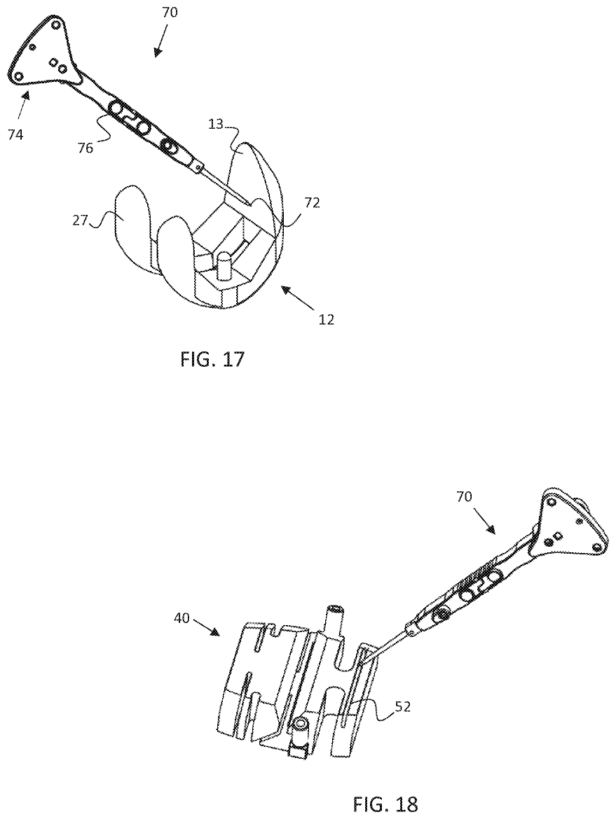

In particular embodiments, with reference to FIG. 17, a digitizer probe 70 is shown being used to digitize surfaces of a femoral implant 12. The digitizer probe 70 includes a probe tip 72, a fiducial marker array 74, and one or more user input mechanisms 76 (e.g., buttons). The digitizer probe 70 is tracked in space by the tracking system by way of the fiducial marker array 74. The fiducial marker array 74 may include three or more fiducial markers (e.g., LEDs, retroreflective spheres) to indicate the position of the digitizer probe 70 to the tracking system. The probe tip 72 is configured to contact another object, such as a surface of the implant 12 or a surface of the bone. The real-time location of the probe tip 72 may be determined by a computing system based on the tracked location of the fiducial marker array 74 and calibration data that defines the location of the probe tip 72 relative to the fiducial marker array 74. The digitizer probe 70 may further include one or more active light-emitting-diodes (LEDs) for optically communicating data to the tracking system. For example, when a user activates the user input mechanism 76 (e.g., pushes a button), a signal is sent to the tracking system via the active LED signaling that the button was pushed. The user input mechanism 76 allows the user to provide a signal to the system to digitize or collect the real-time location of the probe tip 72 and thereby digitize or collect one or more points on the surface of an implant that is in contact with the probe tip 72.

Prior to a TKA procedure, the digitizer probe 70 and a tracking system may be used to determine certain geometry data about an unsupported implant, or geometry data for a plurality of unsupported implants where each implant has a different implant size (e.g., an implant line manufactured by company ‘X’, with implants ranging from size 0 to 10). To do so, a user wielding the digitizer probe may digitize several points on certain surfaces of the implant to determine geometry data of those surfaces, given that the physical implants are available prior to the procedure. In particular, the user may “paint” (e.g., digitize or collect a plurality of points) the contact surfaces (13, 15, 17, 19, 21) of the femoral implant 12 to obtain geometry data about the location of each contact surface relative to the other contact surfaces. For example, a user wielding the digitizer probe 70 may digitize a plurality of points on the anterior contact surface 13, a plurality of points on the anterior chamfer contact surface 15, a plurality of points on the distal contact surface 17, a plurality of points on the posterior chamfer contact surface 19, and a plurality of points on the posterior contact surface 21. The points may be digitized in the manner described above, where the user activates a user-input mechanism when the probe tip 72 is in contact with a contact surface, and the tracking system (or computing system operatively coupled to the tracking system) then records the location of the probe tip 72 at those locations to digitize the contact surfaces. The implant may be tracked by a tracking system (e.g., via a tracking array affixed to the implant) or securely fixed in position while the user digitizes the implant. The plurality of points digitized on each contact surface may be stored in the memory of the computing system. The above procedure may be repeated on different implants of different implant sizes to build a library of geometry data for an entire implant line if so desired. In addition to digitizing the contact surfaces (13, 15, 17, 19, 21) of the implant, the user may further digitize a plurality of points on the outer surface (e.g., 27) of the implant (sometimes referred to as the articulating surface of the implant). Digitizing the outer surface of the implant provides geometry data that allows the user to assess how the outer surfaces of the implant (e.g., femoral implant) will contact, move, or articulate relative to the opposing TKA implant (e.g., tibial liner) during pre-operative or intra-operative planning. Digitizing the outer surface may also provide geometry data that allows the user to assess how the patella might track in the anterior groove of the femoral implant 12 during pre-operative or intra-operative planning. Digitizing the outer surface may further provide geometry data regarding the thickness of the implant (e.g., the posterior thickness of the femoral implant 12).

The plurality of digitized points that represent geometry data about the implant(s) may now be used to assist a user in planning and/or executing a TKA procedure. In one embodiment, the computing system includes planning software for planning the surgical procedure. The planning software may utilize the plurality of digitized points as raw data to assist a user with planning the procedure (e.g., displaying the digitized points as a point cloud showing the relationship of each contact surface relative to the other contact surface, or showing a point cloud of the outer surface of the implant). In other embodiments, the planning software may fit a plane to the plurality of points digitized on each contact surface. For a conventional femoral implant, the software may fit a total of five planes corresponding to the five contact surfaces, where each plane is fitted to a contact surface. The intersection of those planes marks the boundary between one contact surface relative to its neighboring contact surface. The planning software then uses the fitted planes (in their fixed relationship) to assist a user with planning the procedure. In another embodiment, the planning software may utilize statistical shape modeling, morphing, or other 3-D modeling software to generate a 3-D model of the implant from the plurality of digitized points (e.g., fitting the plurality of digitized points to a generic 3-D implant model). The planning software may then use the generated 3-D model to assist a user with planning the procedure.

The planning software may include various software tools that allow the user to position any of the aforementioned (e.g., point cloud, fitted planes, generated 3-D model, hereinafter referred to as digitized implant data) relative to bone data (e.g., 3-D bone model) of the patient's bone to designate the best fit and alignment for the implant on the bone. For example, instead of showing the generic planes 36 or generic implant model 38 as shown in FIG. 4, the planning software can now display the digitized implant data to assist the user in planning the location for the implant relative to the bone data 32. After the user is satisfied with the planned location for the digitized implant data relative to the bone data, the surgical plan may be saved for future use (e.g., used to execute the TKA procedure with the assistance of a CAS device). It will be appreciated that the planned location for the digitized implant data relative to the bone data defines the planned location for forming the cut surfaces as described above. Thus, by digitizing the implant as described herein, a once unsupported implant can now be used with the CAS system in a more traditional manner.

With reference now to FIG. 18, another embodiment for determining implant and/or cut block geometry data is shown for using an unsupported implant with a CAS system. In this embodiment, the user wielding the digitizer digitizes the guide slots of the cut block 40 to determine the locations of the guide slots relative to one another. The user may place the probe tip 72 in a guide slot (e.g., anterior guide slot 520) and slide the digitizer across the guide slot while the tracking system (or a computing system operatively coupled to the tracking system) records the location of the probe tip 72. The user may digitize points in the guide slot on both the top side 44 of the cut block 40 and on the bottom side 45 of the cut block 40 to provide enough digitized points to define both the location and orientation of each guide slot. The cut block 40 may be tracked by a tracking system (e.g., via a tracking array affixed to the cut block 40) or securely fixed in position while the user digitizes the cut block 40. Planning software may use the digitized points to fit a plane to each guide slot, where the intersection of one plane with another plane defines the boundary of one guide slot (e.g., posterior guide slot) to its neighboring guide slot (e.g., posterior chamfer guide slot). The cut block is dimensioned to correspond to the shape of the femoral implant, such that the cut surfaces formed with the aid of the guide slots correspond to the shape of the contact surfaces of the femoral implant. Thus, for a 4-in-1 cut block, digitizing the four guide slots provides the relative locations of the posterior contact surface 21, the posterior chamfer contact surface 19, the anterior chamfer contact surface 15, and the anterior contact surface 13 of the femoral implant 12. The distal contact surface is still unknown, but the location for forming the distal cut surface may be planned using the techniques described with reference to FIG. 4. The planning software may then display the planes that were fitted to the digitized points in the guide slots (where the fitted planes correspond to the contact surfaces of the implant) relative to the bone data to designate the best fit and alignment for the implant on the bone. The user may also digitize the location of the pegs (54a, 54b) of the cut block 40 to determine the offset between the pegs (54a, 54b) and one or more of the guide slots. With all this data, the CAS system may be used in a conventional manner. For example, the system may be a 2-DoF CAS system as described in U.S. Pat. No. 11,457,980, where the location of the pegs (54a, 54b) relative to the planned location for the posterior or anterior cut surface may be used to assist in drilling peg holes in the bone to receive the pegs of the cut block in the planned position and orientation.

Aligning a Cut Block on a Bone using Pins Inserted in the Bone In particular embodiments, with reference to FIGS. 19A-21B , a system and method for aligning a cut block on a bone in a planned POSE is shown, where the cut block is aligned on the bone using alignment pins inserted in the bone. The alignment pins are inserted in the bone coincident with the second virtual plane ‘VP2’ corresponding the location for forming the posterior cut surface. The alignment pins are aligned with the use of a CAS device (e.g., 2-DoF device 100) for inserting the alignment pins in the bone. At least a portion of the alignment pin has a diameter (or width) that allows the posterior guide slot of a cut block to fit over/onto the alignment pin, thereby aligning the posterior guide slot with the planned location for forming the posterior cut surface, and thereby aligning the entire cut block on the bone in the planned POSE for forming the remaining cut surfaces on the bone.

FIGS. 19A and 19B depicts a first embodiment of an alignment pin assembly 80, where FIG. 19A depicts the alignment pin assembly 80 in an assembled state, and FIG. 19B depicts the alignment pin assembly 80 in an exploded state. The alignment pin assembly 80 includes a sleeve 82 and an alignment pin 84. The sleeve 82 is configured to slide over a top portion of the alignment pin 84. The sleeve 82 may be cylindrical having an inner bore or opening for receiving at least the top portion of the alignment pin 84 therein. The sleeve 82 may further include a coupling member 86 (e.g., a square or hexagonal bit) located at a proximal end of the sleeve 82. The coupling member 86 is configured to couple the alignment pin assembly 80 to a CAS device (e.g., 2-DoF device 100) by way of a coupling mechanism (e.g., a chuck, collet, interaction fit, bit and socket connection, etc.). The alignment pin 84 includes a pin tip 88 located at a distal end of the alignment pin 88 to easily pierce the bone when inserting the alignment pin assembly 80 into the bone. The inner bore or opening of the sleeve 82 has a diameter (or width) greater than the diameter (or width) of the alignment pin 84 in order to slide over at least a top portion of the alignment pin 84. In particular embodiments, the outer diameter (or outer width) of the sleeve 82 is dimensioned to match the outer diameter (or outer width) of a typical tool (e.g., pin 107, a drill bit, etc.) driven by the CAS device. More particularly, the coupling member 86 is dimensioned to match the dimensions of a coupling member of a typical tool drive by the CAS device. For example, a tool may be coupled to a CAS device by way of a socket and bit connection, where the tool includes a bit (e.g., coupling member 86) located at its proximal end that fits inside a socket associated with the driving mechanism of the CAS device. Therefore, the dimensions of the socket associated with the CAS device does not need to be modified or changed in order to receive the sleeve 82 and then drive the alignment pin assembly 80 into the bone. One concern is that the alignment pin 84 may have an outer diameter (or outer width) that is less than the dimensions of the socket associated with the CAS device (because the alignment pin needs to fit inside the guide slot of the cut block), and the use and dimensions of the sleeve 82 allow the alignment pin assembly 80 to still couple to the CAS device for driving the alignment pin 84 into the bone. The diameter (or width) of the alignment pin 84 is dimensioned to be less than the width of a guide slot of a cut block such that the alignment pin 84 can snuggly fit inside the guide slot. In other words, the diameter (or width) of the alignment pin 84 is dimensioned such that a guide slot of the cut block can slide over/onto the alignment pin 84 without much play therebetween. In specific embodiments, the alignment pin 84 has a diameter (or width) between 0.5 millimeters and 1.5 millimeters, and the sleeve 82 has an outer diameter (or width) between 2.5 millimeters and 3.5 millimeters with the diameter of the inner bore or opening greater than the diameter of the alignment pin 84.

In another embodiment, a top portion of the alignment pin 84 may be shaped to have a rectangular cross-section to fit inside the inner bore of the sleeve 82, where the inner bore is shaped to have a negatively matching rectangular cross-section. The rectangular cross-sectional fit ensures that the alignment pin 84 does not rotate inside the sleeve 82 when the sleeve 82 is driven and rotated by the CAS device. A bottom portion of the alignment pin 84 may still be cylindrical to be easily inserted into the bone. The top portion of the alignment pin 84 may further have a rough surface or something to be able to grip the alignment pin 84 with a pin puller or with the sleeve 82 in order to pull the alignment pin 84 out of the bone after its use is no longer needed in the bone.

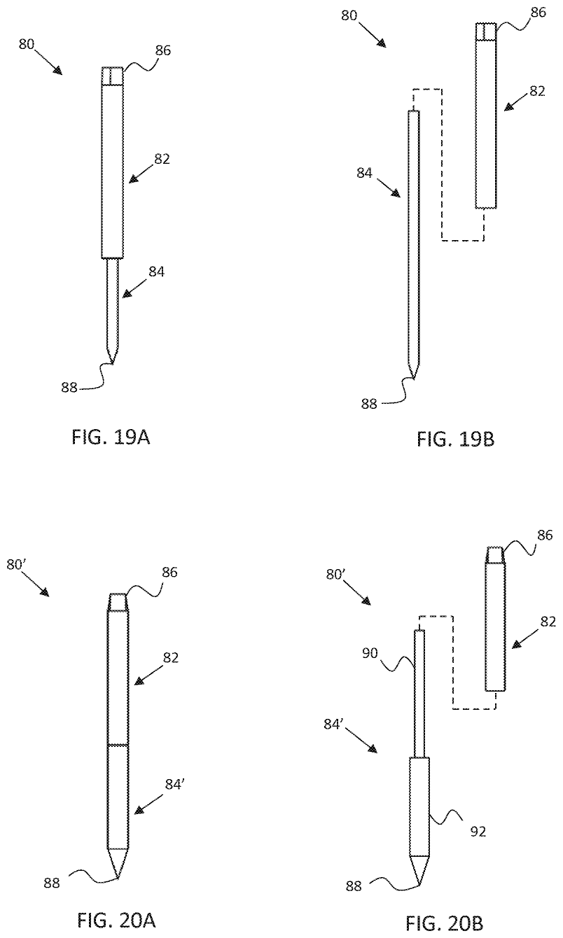

FIGS. 20A and 20B depict a second embodiment of an alignment pin assembly 80′, where FIG. 20A depicts the alignment pin assembly 80′ in an assembled state, and FIG. 20B depicts the alignment pin assembly 80′ in an exploded state. The alignment pin assembly 80′ may include a sleeve 82 and a tiered alignment pin 84′. The sleeve 82 is configured to slide over a top tier 90 of the tiered alignment pin 84′. The sleeve 82 may be cylindrical having an inner bore or opening for receiving the top tier 90 of the alignment pin 84′ therein. The sleeve 82 may further include a coupling member 86 (e.g., a square or hexagonal peg/nut) located at a proximal end of the sleeve 82. The coupling member 86 is configured to couple the alignment pin assembly 80′ to a CAS device (e.g., 2-DoF device 100) by way of a coupling mechanism (e.g., a chuck, collet, interaction fit). The tiered alignment pin 84′ includes a top tier 90 (or top portion) and bottom tier 92 (or bottom portion), where the bottom tier 92 further includes a pin tip 88 located at a distal end of the bottom tier 82 to easily pierce the bone when inserting the alignment pin assembly 80′ into the bone. The inner bore or opening of the sleeve 82 has a diameter (or width) greater than the diameter (or width) of the top tier 90 of the tiered alignment pin 84′, but less than the diameter (or width) of the bottom tier 92, such that the sleeve 82 cannot slide over the bottom tier 92. In particular embodiments, the outer diameter (or outer width) of the sleeve 82 is dimensioned to match the outer diameter (or outer width) of a typical tool (e.g., pin 107, a drill bit, etc.) driven by the CAS device. More particularly, the coupling member 86 is dimensioned to match the dimensions of a coupling member of a typical tool drive by the CAS device. For example, a tool may be coupled to a CAS device by way of a socket and bit connection, where the tool includes a bit (e.g., coupling member 86) located at its proximal end that fits inside a socket associated with the driving mechanism of the CAS device. Therefore, the dimensions of the socket associated with the CAS device does not need to be modified or changed in order to receive the sleeve 82 and then drive the tiered alignment pin assembly into the bone. One concern is that the top tier 90 of the tiered alignment pin 84′ may have an outer diameter (or outer width) that is less than the dimensions of the socket associated with the CAS device (because the top tier of the alignment pin needs to fit inside the guide slot of the cut block), and the use and dimensions of the sleeve 82 allow the alignment pin assembly 80′ to still couple to the CAS device for driving the tiered alignment pin 84′ into the bone. The diameter (or width) of the top tier 90 of the tiered alignment pin 84′ is dimensioned to be less than the width of a guide slot of a cut block such that the top tier 90 can snuggly fit inside the guide slot. In other words, the diameter (or width) of the top tier 90 is dimensioned such that a guide slot of the cut block can slide over/onto the alignment pin 84 without much play therebetween. In specific embodiments, the top tier 90 has a diameter (or width) between 0.5 millimeters and 1.5 millimeters, the bottom tier 92 has a diameter (or width) between 2.5 millimeters and 3.5 millimeters, and the sleeve 82 has an outer diameter (or width) between 2.5 millimeters and 3.5 millimeters (which may be configured to substantially match the diameter of the bottom tier) with the diameter of the inner bore or opening greater than the diameter of the top tier but less than the diameter of the bottom tier.

In another embodiment, at least a top portion of the top tier 90 may be shaped to have a rectangular cross-section to fit inside the inner bore of the sleeve 82, where the inner bore is shaped to have a negatively matching rectangular cross-section for the reasons previously described. The top tier 90 of the tiered alignment pin 84′ may further have a rough surface or something to be able to grip the tiered alignment pin 84′ with a pin puller or with the sleeve 82 in order to pull the alignment pin 84 out of the bone after its use is no longer needed in the bone.

One advantage of the alignment pin assembly 80 as shown in FIGS. 19A and 19B is that the diameter (or width) of the portion of the alignment pin 84 that is inserted into the bone is less than the width of the guide slot (and the surgical saw blade). Therefore, when the user forms the proximal cut surface in the bone with a saw blade, the hole formed by the alignment pin 84 is completely removed. Inserting the alignment pin 84 in the bone may also be easier due to its smaller diameter. However, since the diameter of the alignment pin 84 is small, it may bend or break, so preferably, the alignment pin 84 is made of a strong material such as carbide. One advantage of the alignment pin assembly 80′ shown in FIGS. 20A and 20B is that the portion of the tiered alignment pin 84′ (i.e., the bottom tier 92) inserted into the bone has a larger diameter and likely won't bend or break. The bottom tier 92 is also easily inserted into the bone. However, since the bottom tier 92 has a diameter greater than the width of the guide slot of the cut block (and it's this bottom tier 92 that is getting inserted into the bone), there will be a ding left in the bone on the side of the posterior cut surface after the user has formed the posterior cut surface with the saw blade. This may or may not be clinically relevant, but the user may have a preference of using the first alignment pin assembly 80 vs. the second pin alignment assembly 80′.

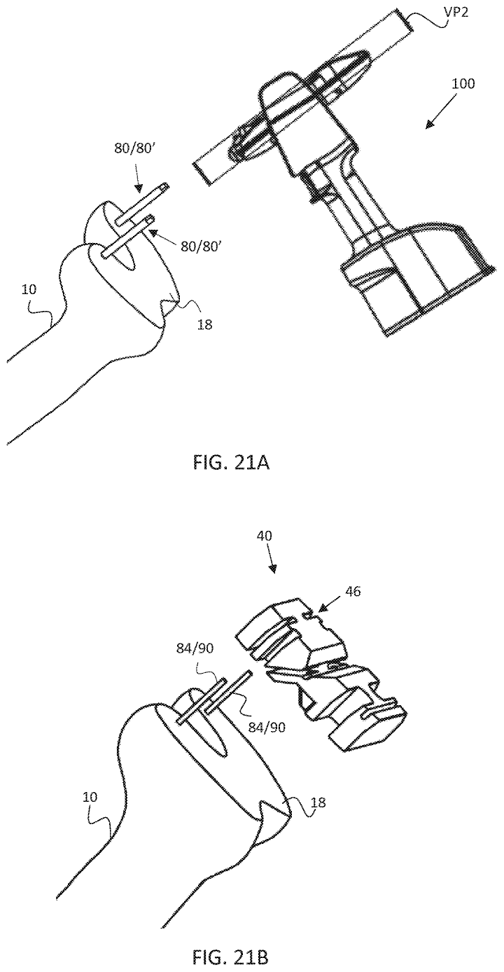

FIGS. 21A and 21B depict a system and method for inserting the pin alignment assembly (80 or 80′) in the bone for aligning a cut block on the bone in a planned POSE. It should be appreciated that the system and methods for planning the placement of an implant relative to a bone may be the same as previously described with reference to FIG. 4 and FIG. 5. After planning, a CAS device may be used to aid in the formation of the distal cut surface. For example, with reference back to FIGS. 6 and 7, a 2-DoF device 100 may be used to place pins (107a, 107b) in the bone coincident with a first virtual plane ‘VP.’ A cut guide 108 is then placed on the pins (107a, 107b) and a user advances a surgical saw blade through the guide slot 112 to form the distal cut surface 18. After the distal cut surface 18 is formed, a CAS device is used to insert alignment pin assemblies (80 or 80′) in the bone coincident with the location for forming the posterior cut surface 22. For example, as shown in FIG. 21A, a 2-DoF device 100 is used to maintain alignment of an alignment pin assembly (80 or 80′) coincident with a second virtual plane ‘VP2’ for inserting the alignment pin assemblies (80 or 80′) in the bone (and more specifically, into the distal cut surface of the bone) coincident with the second virtual plane ‘VP2’. Here, the location of the second virtual plane ‘VP2’ corresponds to the location for forming the posterior cut surface 22 on the bone. The user may insert two or more alignment pin assemblies (80 or 80′) into the bone. After the alignment pin assemblies (80 or 80′) are inserted in the bone, the sleeves 82 are removed from the alignment pin assemblies (80 or 80′) revealing either the alignment pin 84 or the top tier 90 of the tiered alignment pin 84′. The user may then place the posterior guide slot 46 of the cut block 40 onto the alignment pin 84 or top tier 90 of the tier alignment pin 84′. For example, the user may slide the posterior guide slot 46 over/onto two alignment pins 84 (or top tiers 90) inserted in the bone. This aligns the posterior guide slot with the location for forming the posterior cut surface 22, and thereby aligns the entire cut block 40 on the bone in the planned POSE. The user may push the cut block 40 into the distal cut surface 18 (while the posterior guide slot is positioned on the alignment pin 84 or top tier 90) such that the pegs (54a, 54b) of the cut block mark (e.g., form a depression in the bone) the location for forming the peg holes in the distal cut surface in the correct location. After the location for the peg holes is marked, the user may remove the alignment pin 84 or tiered alignment pin 90 from the bone and drill the peg holes in the distal cut surface at the marked location. The user may then place the pegs (54a, 54b) of the cut block 40 in the formed peg holes and proceed to form the remaining cut surfaces in the bone by advancing a saw blade through the guide slots in the cut block 40. After the cut surfaces are formed, the user can install the implant in a conventional manner.

It should be appreciated that instead of using alignment pin assemblies for aligning the cut block on the bone, an alignment pin having a diameter (or width) less than the guide slot may be used without the sleeve 82 or having tiers. For example, the 2-DoF device may couple with and drive just the alignment pin 84 for inserting the alignment pin 84 into the bone. In other words, the sleeve 82 and tiers may be optional. In this embodiment, the alignment pin 84 may have a coupling member (e.g., like the coupling member 86) located at its proximal end for directly coupling the alignment pin 84 to a CAS device.

### Computer-Assisted Surgical (CAS) System

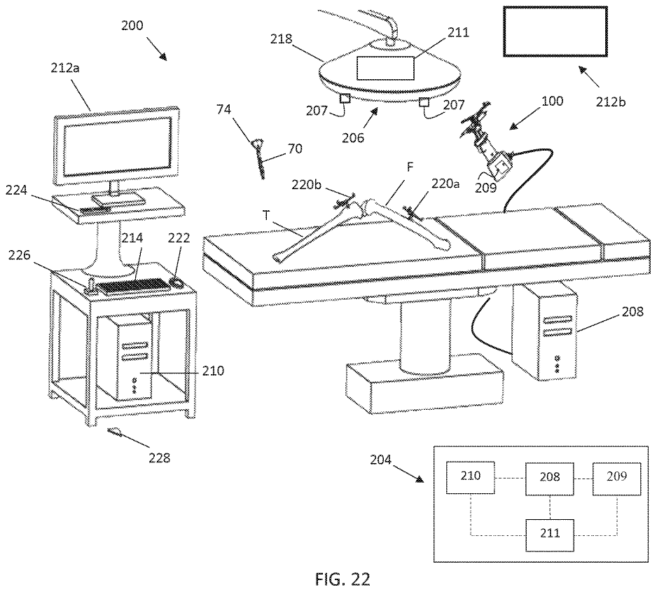

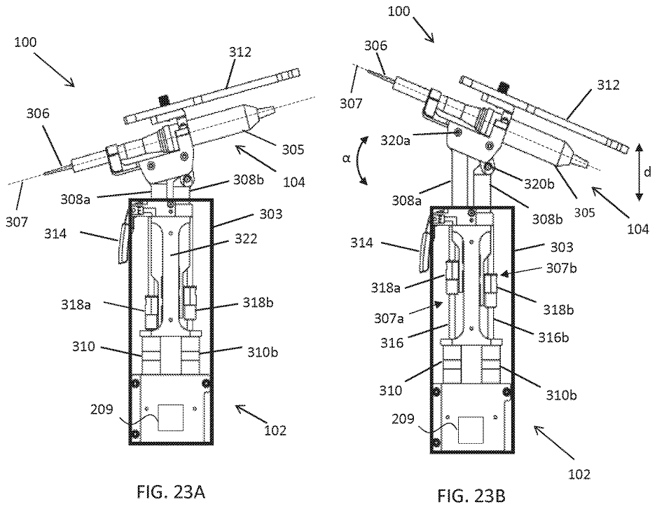

Referring now to FIGS. 22, 23A, and 23B, embodiments of the present inventive system and method generally includes a computer-assisted surgical system. In some inventive embodiments, a 2-DoF device 100 is provided for maintaining alignment of an end-effector 206 (e.g., bone pin) coincident with a virtual plane. FIG. 22 is a schematic view showing the computer-assisted surgical system 200 including a 2-DoF device 100, a computing system 204, and a tracking system 206. In other inventive embodiments, the system includes an end effector 306 extending from a robotic arm.

The computing system 204 generally includes hardware and software for executing a surgical procedure. By way of example but not limitation, in one preferred form of the present invention, the computing system 204 is configured to control the actuation of the working portion 104 relative to the hand-held portion 102 of the 2-DoF 100 device to maintain alignment of the end-effector axis 307 (FIG. 23A) coincident with a virtual plane defined in a surgical plan. The end-effector 206 coupled to the working portion 104 in operation modifies (e.g., inserts pins, cuts, mills, etc.) subject bone. The computing system 204 may generate control signals to accurately maintain the end-effector axis 207 coincident with a virtual plane defined in the surgical plan based on: a) the location of the virtual plane as registered to the location of the bone (or more specifically to the coordinate system of a tracking array affixed to the bone); and b) the tracked POSE of the 2-DoF device 100.

The computing system 204 of the computer-assisted surgical system 200 may include: one or more device computers (208, 209) including a planning computer 210; a tracking computer 211, and peripheral devices. Each computer may include one or more processors. Processors operate in the computing system 204 to perform computations and execute software associated with the inventive system and method. The device computer(s) (208, 209), the planning computer 210, and the tracking computer 211 may be separate entities as shown in FIG. 22, or it is also contemplated that operations may be executed on one (or more) computers depending on the configuration of the computer-assisted surgical system 200. For example, the tracking computer 211 may have operational data to control the 2-DoF device 100 without the need for a device computer (208, 209). Furthermore, if desired, any combination of the device computers (208, 209), planning computer 210, and/or tracking computer 211 may be connected together via a wired or wireless connection. It is further appreciated that one or more of the computers may be readily located remote from the surgical site. Cloud-based computation is also contemplated in the present invention. In addition, the data gathered by, and/or the operations performed by, the tracking computer 211 and device computer(s) (208, 209) may work together to control the 2-DoF device 100 and, as such, the data gathered by, and/or the operations performed by, the tracking computer 211 and device computer(s) (208, 209) to control the 2-DoF device 100 may be referred to herein as a “control system.”

The peripheral devices allow a user to interface with the computing system 204 and may include, but are not limited to, one or more of the following: one or more user-interfaces, such as a display or monitor (212a, 212b) to display a graphical user interface (GUI); and user-input mechanisms, such as a keyboard 214, mouse 222, pendent 224, joystick 226, and foot pedal 228. If desired, the monitor(s) (212a, 212b) may have touchscreen capabilities, and/or the 2-DoF device 100 may include one or more input mechanisms (e.g., buttons, switches, etc.). Another peripheral device may include a tracked digitizer probe 70 to assist in the registration process, or to digitize portions of an implant or cut guide/block. A tracking array 74 is assembled to the digitizer probe 70 to permit the tracking system 206 to track the POSE of the digitizer probe 70 in space. The digitizer probe 70 may further include one or more user input mechanisms 76 to provide input to the computing system 204. For example, a button on the digitizer probe 70 may allow the user to signal to the computing system 204 to digitize a point in space to assist in registering a bone to a surgical plan.

The device computer(s) (208, 209) may include one or more processors, controllers, software, data, utilities, and/or storage medium(s) such as RAM, ROM or other non-volatile or volatile memory to perform functions related to the operation of the 2-DoF device 100. By way of example but not limitation, one or more of the device computers (208, 209) may include software to control the 2-DoF device 202, e.g., generate control signals for the actuators to move the working portion 104 relative to the hand-held portion 102 to a targeted POSE, receive and process tracking data, control the rotational or oscillating speed of the end-effector 306 by controlling motor 305, execute registration algorithms, execute calibration routines, provide workflow instructions to the user throughout a medical procedure, as well as any other suitable software, data or utilities required to successfully perform the procedure in accordance with embodiments of the invention.

In some embodiments, the system 200 may include a first device computer 208 located separate from the 2-DoF device 100 and a second device computer 209 housed in the 2-DoF device 100 to provide on-board control. The first device computer 208 may be dedicated to the control of the surgical workflow via a GUI, the registration process and the associated calculations, the display of 3-D models and 3-D model manipulation or animation, as well as other processes. The second device computer 209, also referred to herein as an on-board device computer, may be dedicated to the control of the 2-DoF device 100. For example, the on-board device computer 209 may compute and generate the control signals for the actuator motors (210a, 210b) based on: i) received signals/data corresponding to the real-time POSE of the 2-DoF device 100 from the tracking system; and ii) received signals/data corresponding to the real-time POSE of the virtual plane computed by first device computer 208. The on-board device computer 209 may also send internal data (e.g., operational data, actuator/screw position data, battery life, etc.) via a wired or wireless connection. In some inventive embodiments, wireless optical communication is used to send and receive the signals/data described herein. Details about bi-directional optical communication between a 2-DoF device 100 and a tracking system 206 are further described below.

The planning computer 210 in some inventive embodiments is dedicated to planning the procedure. By way of example but not limitation, the planning computer 210 may contain hardware (e.g., processors, controllers, memory, etc.), planning software, data, and/or utilities capable of: receiving, reading, and/or manipulating medical imaging data; segmenting imaging data; constructing and manipulating three-dimensional (3D) virtual bone models; storing and providing computer-aided design (CAD) files such as implant CAD files; planning the POSE for cut surfaces, virtual planes, screws, pins, implants, grafts, and fixation hardware relative to pre-operative bone data; generating the surgical planning data for use with the system 200, and providing other various functions to aid a user in planning the surgical procedure. The planning software is particularly programmed for planning TKA procedures for an implant agnostic system as described above. The final surgical plan data may include one or more images of a bone or virtual models of the bone, registration data, subject identification information, the POSE for inserting or mounting one or more pins, screws, implants, grafts, fixation hardware relative to the bone, and/or the POSE of one or more virtual planes defined relative to the bone models. The device computer(s) (208, 209) and the planning computer 210 may be directly connected in the operating room, or the planning computer 210 may exist as separate entities outside the operating room. The final surgical plan is readily transferred to a device computer (208, 209) and/or tracking computer 211 through a wired (e.g., electrical connection) or a wireless connection (e.g., optical communication) in the operating room; or transferred via a non-transient data storage medium (e.g., a compact disc (CD), or a portable universal serial bus (USB drive)) if the planning computer 210 is located outside the operating room (or if otherwise desired). As described above, the computing system 204 may comprise one or more computers, with multiple processors capable of performing the functions of the device computer 208, the tracking computer 211, the planning computer 210, or any combination thereof.

The tracking system 206 of the present invention generally includes a detection device to determine the POSE of an object relative to the position of the detection device. In particular embodiments of the present invention, the tracking system 206 is an optical tracking system such as the optical tracking system described in U.S. Pat. No. 6,061,644 (which patent is hereby incorporated herein by reference), having two or more optical detectors 207 (e.g., cameras) for detecting the position of fiducial markers arranged on rigid bodies or integrated directly on the tracked object. By way of example but not limitation, the fiducial markers may include an active transmitter, such as an LED or electromagnetic radiation emitter; a passive reflector, such as a plastic sphere with a retroreflective film; or a distinct pattern or sequence of shapes, lines or other characters. A set of fiducial markers arranged on a rigid body, or integrated on a device, is sometimes referred to herein as a tracking array (220a, 220b, 74, 312), where each tracking array has a unique geometry/arrangement of fiducial markers, or a unique transmitting wavelength/frequency (if the markers are active LEDS), such that the tracking system 206 can distinguish between each of the tracked objects.

If desired, the tracking system 206 may be incorporated into an operating room light 218, located on a boom, a stand, or built into the walls or ceilings of the operating room. The tracking system computer 211 includes tracking hardware, software, data, and/or utilities to determine the POSE of objects (e.g., tissue structures, the 2-DoF device 100) in a local or global coordinate frame. The output from the tracking system 206 (i.e., the POSE of the objects in 3-D space) is referred to herein as tracking data, where this tracking data may be readily communicated to the device computer(s) (208, 209) through a wired or wireless connection. In a particular embodiment, the tracking computer 206 processes the tracking data and provides control signals directly to the 2-DoF device 100 and/or device computer 208 based on the processed tracking data to control the position of the working portion 104 of the 2-DoF device 100 relative to the hand-held portion 102. In another embodiment, the tracking computer 206 sends tracking data to a receiver located on the 2-DoF device 100, where an on-board device computer 209 generates control signals based on the received tracking data.

The tracking data is determined in some inventive embodiments using the position of the fiducial markers detected from the optical detectors and operations/processes such as image processing, image filtering, triangulation algorithms, geometric relationship processing, registration algorithms, calibration algorithms, and coordinate transformation processing. These operations/processes may be executed directly on the tracking system computer 211 or executed on a separate computer (e.g., first device computer 208) in communication with the tracking system 206.

Bi-directional optical communication (e.g., light fidelity or Li-Fi) may occur between the 2-DoF device 100 and the tracking system 206 by way of a modulated light source (e.g., light emitting diode (LED)) and a photosensor (e.g., photodiode, camera). The 2-DoF device 100 may include an LED and a photosensor (i.e., a receiver) disposed on the working portion 104 or hand-held portion 102, where the LED and photosensor are in communication with a processor such as modem or an on-board device computer. Data generated internally by the 2-DoF device 100 may be sent to the tracking system 206 by modulating the LED, where the light signals (e.g., infrared, visible light) created by the modulation of the LED are detected by the tracking system optical detectors (e.g., cameras) or a dedicated photosensor and processed by the tracking system computer 211. The tracking system 206 may likewise send data to the 2-DoF device 100 with a modulated LED associated with the tracking system 206. Data generated by the tracking system 206 may be sent to the 2-DoF device 202 by modulating the LED on the tracking system 206, where the light signals are detected by the photosensor on the 2-DoF device 100 and processed by a processor in the 2-DoF device 100. Examples of data sent from the tracking system 206 to the 2-DoF device 100 includes operational data, surgical planning data, informational data, control data, positional or tracking data, pre-operative bone data, or instructional data. Examples of data sent from the 2-DoF device 100 to the tracking system 206 may include motor position data, battery life, operating status, logged data, operating parameters, warnings, or faults. In some embodiments, data generated by the first device computer 208 is sent to the tracking system 206, where that generated data is transferred to the 2-DoF device 100 via the LED on the tracking system 206.

It should be appreciated that in some embodiments of the present invention, other tracking systems are incorporated with the surgical system 200. By way of example but not limitation, the surgical system 200 may comprise an electromagnetic field tracking system, ultrasound tracking systems, accelerometers and gyroscopes, and/or a mechanical tracking system. The replacement of a non-mechanical tracking system with other tracking systems will be apparent to one skilled in the art in view of the present disclosure. In one form of the present invention, the use of a mechanical tracking system may be advantageous depending on the type of surgical system used such as the computer-assisted surgical system described in U.S. Pat. No. 6,322,567 assigned to the assignee of the present application and incorporated herein by reference in its entirety.

FIGS. 23A and 23B are schematic views showing the 2-DoF device 100 in greater detail. More particularly, FIG. 23A shows the 2-DOF device 100 in a first working POSE, and FIG. 23B illustrates the 2-DOF device 100 in a second working POSE. The 2-DoF device 100 comprises a hand-held portion 102 (or handle) and a working portion 104. The hand-held portion 102 comprises an outer casing 303 of ergonomic design which can be held and wielded by a user (e.g., a surgeon). In particular embodiments, the 2-DoF device 100 is intended to be fully supported by the hands of the user in that there are no additional supporting links connected to the 2-DoF device 100 and the user supports the full weight of the 2-DoF device 100. The working portion 104 comprises an end-effector 306 having an end-effector axis 307. The end-effector 306 may be removably coupled to the working portion 104 (via a coupler (e.g., chuck)) and driven by a motor 305. The hand-held portion 102 and working portion 104 are connected to one another, for example, by a first linear actuator 307a and a second linear actuator 307b in order to control the pitch and translation of the working portion 104 relative to the hand-held portion 102, as will hereinafter be discussed in further detail. In a particular embodiment, the working portion 104 is removably coupled to the hand-held portion 102 to permit different types of working portions to be assembled to the hand-held portion 102. For example, a first working portion 104 may illustratively be a laser system having components to operate a laser for treating tissue, a second working portion 104 may illustratively be a drill for rotating a bone pin, and a third working portion 104 may illustratively be an oscillating saw.

A tracking array 312, having three or more fiducial markers of the sort well known in the art, is preferably rigidly attached to the working portion 104 in order to permit the tracking system 206 (FIG. 22) to track the POSE of the working portion 104. The three or more fiducial markers may, alternatively, be integrated directly with the working portion 104. The fiducial markers may be active markers such as light emitting diodes (LEDs), or passive markers such as retroreflective spheres. The 2-DoF device 100 may further include one or more user input mechanisms such as triggers (e.g., trigger 314) or button(s). The user input mechanisms may permit the user to perform various functions illustratively including: activating or deactivating the motor 305; activating or deactivating the actuation of the working portion 104 relative to the hand-held portion 102; notifying the computing system 204 to change from targeting one virtual plane to a subsequent virtual plane; and pausing the surgical procedure.

Within the outer casing of the hand-held portion 102 is the a linear actuator 307a and a second linear actuator 307b. Each linear actuator (307a, 307b) may include a motor (310a, 310b) to power a screw (316a, 316b) (e.g., a lead screw, a ball screw), a nut (318a, 318b), and a linear member (308a, 308b). In some inventive embodiments, the motors (first motor 310a, second motor 310b) are electric servo-motors that bi-directionally rotate the screws (316a, 316b). Motors (310a, 310b) may also be referred to herein as linear actuator motors. The nuts (318a, 318b) (e.g., ball nuts, elongated nuts) are operatively coupled to the screws (316a, 316b) to translate along the screws (316a, 316b) as each screw is rotated by its respective motor (310a, 310b). A first end of each linear member (308a, 308b) is coupled to a corresponding nut (316a, 316b) and the opposing end of each linear member (308a, 308b) is coupled to the working portion 104 via hinges/links (320a, 320b), such that the hinges/links (320a, 320b) allow the working portion 104 to pivot relative to the linear rails (308a, 308b). The motors (310a, 310b) power the screws (316a, 316b) which in turn cause the nuts (318a, 318b) to translate along the axis of the screws (316a, 316b). Translation of nuts 318a, 318b along ball screws 316a, 316b, respectively, causes translation of front linear member 308a and back linear member 308b, respectively, whereby to permit (a) selective linear movement of working portion 104 relative to hand-held portion 102, and (b) selective pivoting of working portion 304 relative to hand-held portion 302 of 2-DoF device 100. Accordingly, the translation “d” and pitch “a” (FIG. 23B) of the working portion 104 may be adjusted depending on the position of each nut (318a, 318b) on their corresponding screw (316a, 316b). A linear guide 322 (FIG. 23A) may further constrain and guide the motion of the linear member (308a, 308b) in the translational direction “d”. In a particular embodiment, the nuts (316a, 316b) are elongated and couple directly to the working portion 104 via the hinges/links (320a, 320b), in which case the linear member (308a, 308b) are no longer a component of the linear actuators (307a, 307b). It should be appreciated that other linear actuation mechanisms/components may be used to adjust the POSE of the working portion 104 relative to the hand-held portion 102 such as linear motors, pneumatic motors, worm drives and gears, rack and pinion gears, and other arrangements of motors and transmissions.

The 2-DoF device 100 may receive power via an input/output port (e.g., from an external power source) and/or from on-board batteries (not shown).

The motors (305, 310a, 310b) of the 2-DoF device 100 may be controlled using a variety of methods. By way of example but not limitation, according to one method of the present invention, control signals may be provided via an electrical connection to an input/output port. By way of further example but not limitation, according to another method of the present invention, control signals are communicated to the 2-DoF device 100 via a wireless connection, thereby eliminating the need for electrical wiring. The wireless connection may be made via optical communication. In certain inventive embodiments, the 2-DoF device 100 includes a receiver for receiving control signals from the computing system 204 (FIG. 22). The receiver may be, for example, an input port for a wired connection (e.g., Ethernet port, serial port), a transmitter, a modem, a wireless receiver (e.g., Wi-Fi receiver, Bluetooth® receiver, a radiofrequency receiver, an optical receiver (e.g., photosensor, photodiode, camera)), or a combination thereof. The receiver may send control signals from the computing system 204 directly to the motors (305, 310a, 310b) of the 2-DoF device 100, or the receiver may be in communication with a computer (e.g., an on-board device computer 209 as further described below) that processes signals received by the receiver and then generates the control signals for the motors (305, 310a, 310b) based on the received signals.

The advantages of the present invention allow the use of computer-assisted surgery to form the more crucial bone cuts that affect clinical outcomes (e.g., the distal cut surface, the posterior cut surface, and the proximal tibial cut surface), while further allowing the CAS system to be used with unsupported implants.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

1. A method for computer-assisted surgery comprising:
providing a spacer specifically configured for placement on a posterior cut surface to replace removed posterior condyles needed to mount an implant manufacture's sizing guide on a distal cut surface for a total knee arthroplasty (TKA) procedure;
using a computer-assisted surgical (CAS) device to assist in forming the distal cut surface and the posterior cut surface on a femur bone for a total knee arthroplasty (TKA) procedure, wherein forming the posterior cut surface removes the posterior condyles needed for sizing an implant with an implant manufacture's sizing guide; and
placing the spacer on the posterior cut surface to replace the removed posterior condyles in order to mount the implant manufacturer's sizing guide on the distal cut surface for the total knee arthroplasty (TKA) procedure.

2. The method of claim 1, further comprising instructing a user to mount the implant manufacturer's sizing guide on the distal cut surface with the spacer in place.

3. The method of claim 2, further comprising:
using the mounted implant manufacturer's sizing guide to determine an implant size from a plurality of manufacturer implant sizes;
using holes in the mounted implant manufacturer's sizing guide to insert pins or drill holes in the bone; and
using the pins or holes to align a 4-in-1 cut block in a correct POSE to form remaining cut surfaces to accommodate the determined implant size.

4. The method of claim 1, wherein the spacer is an adjustable thickness spacer, optionally wherein the thickness of the spacer is adjustable between at least 8 mm and 11 mm and/or the thickness of the spacer is adjustable in 0.5 mm increments.

5. (canceled)

6. (canceled)

7. The method of claim 1, wherein the spacer is a fixed thickness spacer.

8. The method of claim 1, wherein the thickness of the spacer is equal to or adjustable to at least one of a posterior thickness of a femoral implant or the thickness of the removed posterior condyles.

9. (canceled)

10. The method of claim 1 wherein the CAS device assists in the formation of the distal cut surface and the posterior cut surface without having implant geometry data for the implant.

11. A system for computer-assisted surgery comprising:
a computer-assisted surgical (CAS) device for assisting in the formation of a distal cut surface and a posterior cut surface on a femur bone during a total knee arthroplasty (TKA) procedure, wherein forming the posterior cut surface removes the posterior condyles needed for sizing an implant with an implant manufacture's sizing guide; and
a spacer specifically configured for placement on the posterior cut surface to replace the removed posterior condyles in order to mount the implant manufacturer's sizing guide on the distal cut surface for the total knee arthroplasty (TKA) procedure.

12. The system of claim 11 further comprising the implant manufacturer's sizing guide for mounting to the distal cut surface with the spacer in place.

13. The system of claim 12 wherein the implant manufacturer's sizing guide is used to determine an implant size from a plurality of manufacturer implant sizes.

14. The system of claim 12 wherein the implant manufacturer's sizing guide comprises guide holes for guiding the placement of pins in the bone or for drilling holes in the bone, wherein the pins or holes are used to align a 4-in-1 cut block in a correct POSE to form remaining cut surfaces to accommodate the determined implant size.

15. The system of claim 11, wherein the spacer is an adjustable thickness spacer, optionally wherein the thickness of the spacer is adjustable between at least 8 mm and 11 mm and/or the thickness of the spacer is adjustable in 0.5 mm increments.

16. (canceled)

17. (canceled)

18. The system of claim 11, wherein the spacer is a fixed thickness spacer.

19. The system of claim 11, wherein the thickness of the spacer is equal to or adjustable to at least one of a posterior thickness of a femoral implant or the thickness of the removed posterior condyles.

20. (canceled)

21. The system of claim 11, wherein the CAS device assists in the formation of the distal cut surface and the posterior cut surface without having implant geometry data for the implant.

22. A spacer specifically configured for placement on a posterior cut surface to replace removed posterior condyles needed to mount an implant manufacture's sizing guide on a distal cut surface for a total knee arthroplasty (TKA) procedure.

23. The spacer of claim 22, wherein the spacer is an adjustable thickness spacer, optionally wherein the thickness of the spacer is adjustable between at least 8 mm and 11 mm and/or the thickness of the spacer is adjustable in 0.5 mm increments.

24. (canceled)

25. (canceled)

26. The spacer of claim 22, wherein the spacer is a fixed thickness spacer.

27. The spacer of claim 22, wherein the thickness of the spacer is equal to or adjustable to at least one of a posterior thickness of a femoral implant or the thickness of the removed posterior condyles.

28. (canceled)

29. The spacer of claim 22, wherein the spacer is configured to by referenced by paddles or other posterior condyle referencing feature of the sizing guide.

30-48. (canceled)
